(19) United States
(12) Patent Application Publication
(10) Pub. No.: US 2010/0111908 A1
(43) Pub. Date: May 6, 2010

(54) Induction of Renal Cells for Treatment of Kidney Disease

(76) Inventors: Fangming Lin, Dallas, TX (US); Ling Li, Dallas, TX (US); Peter Igarashi, Plano, TX (US)

Correspondence Address: FULBRIGHT & JAWORSKI L.L.P., 600 CONGRESS AVE., SUITE 2400, AUSTIN, TX, 78701, US

(21) Appl. No.: 12/610,623

(22) Filed: Nov. 2, 2009

Related U.S. Application Data
(60) Provisional application No. 61/110,843, filed on Nov. 3, 2008.

(51) Int. Cl. A61K 35/23; C12N 5/02; C12M 1/00

(52) U.S. Cl. 424/93.7; 435/375; 435/325; 435/289.1

(57) ABSTRACT

Methods and systems for the generation of induced renal cells are provided. For example, methods for inducing renal cells from lineage undifferentiated hematopoietic stem and/or progenitor cell incubated with growth factors are described. Furthermore, the invention provides methods for using induced renal cells for treating a renal disease and/or injury.

The present application claims benefit of priority to U.S. Provisional Application Ser. No. 61/110,843, filed Nov. 3, 2008, the entire contents of which are hereby incorporated by reference.

### BACKGROUND OF THE INVENTION

1. Field of the Invention

The present invention relates generally to the field of stem cell technology. More particularly, it concerns methods and compositions for the production of induced renal cells from hematopoietic progenitor, and/or stem cells for treating renal diseases.

2. Description of Related Art

Kidney disease is a major health problem in the United States, afflicting nearly eight million Americans. Kidney and urinary tract diseases together affect an estimated 20 million people, causing more than 95,000 deaths a year and contributing to an additional quarter of a million. Kidney disorders run the gamut from minor infections to total kidney failure, and can cause fluid and electrolyte disturbance, high blood pressure, anemia, bone metabolic disease, elevated cholesterol and heart disease. When chronic, it can lead to depression and sexual dysfunction.

There are many different kinds of kidney diseases. A disease of the kidney may be a short-term problem, and in this case might not cause permanent kidney damage. Examples include some kidney infections and obstruction from kidney stones, sepsis, hypovolumia, trauma, and some medications can also cause temporary changes in kidney function.

“Acute kidney injury” or “acute renal failure” is a sudden or rapid loss of kidney function. Acute failure may be reversed, or it may sometimes lead to permanent loss of kidney function. More often, diseases that affect the kidney are chronic problems. “Chronic renal failure” is a loss of kidney function that occurs gradually and is often “silent,” going undetected for months or years. In this case, once it is detected, kidney function may be monitored by periodic blood or urine tests from year to year. Examples of chronic diseases that cause kidney damage over many years are high blood pressure, diabetes, and polycystic kidney disease. When the kidneys permanently lose ninety percent or more of their function, a person is diagnosed with “end-stage renal disease (ESRD).”

Acute kidney injury (AKI) carries high morbidity and mortality. The mortality of AKI in humans remains between 50-80% (Schrier et al., 2004; Liano and Pascual, 1998). Moreover, the incidence of AKI is increasing (Xue et al., 2006). The only treatment that is available for AKI consists of supportive measures while waiting for kidney function to return. However, renal recovery is often incomplete and severe long-term sequelae include hypertension and end-stage renal disease. The incident end-stage renal disease population yearly numbers over 100,00 (excluding those with unknown modality). Current treatments for end-kidney disease including chronic dialysis and kidney transplantation comprise 20% of Medicare expenditures and 1.6% of the total health care cost per year in the United States. Treatments for kidney disease such as dialysis and organ transplantation are used as a last resort and present serious risks. Therefore, improved treatment options for treating kidney disease or injury are urgently needed.

### SUMMARY OF THE INVENTION

The present invention overcomes a major deficiency in the art by providing methods and systems to generate an induced renal cell from lineage an undifferentiated hematopoietic stem and/or progenitor cell with enhanced renal conversion efficiency and therapeutic potential. In a first embodiment, there is provided a method of generating an induced renal cell comprising the steps of (a) obtaining a lineage undifferentiated hematopoietic stem and/or progenitor cell; and (b) incubating the hematopoietic stem and/or progenitor cell for a period sufficient to produce an induced renal cell in the presence of (i) at least a histone deacetylase (HDAC) inhibitor, (ii) at least one nephrogenic factor selected from the group consisting of a first agent capable of activating a retinoic acid pathway, a second agent capable of activating an activin A pathway, and a third agent capable of activating a BMP7 pathway, and, (iii) one or more epithelial growth factors, such as epithelial growth factor (EGF), hepatocyte growth factor (HGF) and insulin-like growth factor-1 (IGF-1).

The lineage undifferentiated stem and/or progenitor cell may have a hematopoietic origin, for example, an umbilical cord stem cell, or may be more primitive, such as an embryonic stem cell or an induced pluripotent stem cell.

In certain embodiments, the nephrogenic factor comprises at least two or three of the group consisting of the first agent, the second agent and the third agent. For example, the first agent may be retinoic acid; the second agent may be activin-A; the third agent may be BMP7. In particular, the nephrogenic factor may comprise retinoic acid, activin-A and BMP7. Any nephrogenic factor that may be used to induce a renal fate of the undifferentiated cell is contemplated in the present invention.

In still further aspects of the invention, the HDAC inhibitor may be used to activate renal gene expression to facilitate renal differentiation. The HDAC inhibitor may be selected from the group consisting of a small molecule HDAC inhibitor, a HDAC siRNA, and a HDAC antibody. Non-limiting examples of HDAC inhibitor include hydroxamic acid (e.g., trichostatin A (TSA), vorinostat or suberoylanilide hydroxamic acid (SAHA), CBHA, belinostat/PXD-101, ITF2357 and LBH-589), cyclic peptide (e.g., PCI-24781 and depsipeptide (e.g., FK-228)), benzamide (e.g., MS-275, CI-994 and MGCD0103), electrophilic ketone, and aliphatic acid (e.g., valproic acid, butyrate, phenylbutyrate and AN-9). Particularly, the HDAC inhibitor may be TSA. In certain aspects, a HDAC7 siRNA and/or a HDAC9 siRNA may be used because HDAC7 and HDAC9 expression decreases after renal differentiation and may be involved in renal cell differentiation.

In some further aspects, the epithelial growth factor may be any growth factors that stimulate epithelial cell growth, such as epidermal growth factor (EGF), insulin-like growth factor 1 (IGF-1), hepatocyte growth factor (HGF) or a combination thereof.

In certain aspects, the method may further comprise incubating the hematopoietic stem or progenitor cell with at least a cytokine, such as IL-3, IL-6 and stem cell factor or a combination thereof.

In a further aspect, the method further comprises a step (c) of isolating an induced renal cell. The isolation may be based on renal cell markers or renal epithelial cell characteristics known in the art.

In a still further aspect, the lineage undifferentiated stem cell and/or progenitor cell or the isolated induced renal cell may be incubated with a calcium-sensing receptor (CaR) inhibitor to increase the transmigration to the kidney and decrease homing to the bone marrow. For example, the CaR inhibitor may be a CaR antibody, a CaR siRNA or a small molecule CaR inhibitor (e.g., calcilytics).

The incubation period with the factors described above may be from about 4 hr to about 2 weeks, from about 4 hr to about 1 week, from about 8 hr to about 72 hr, from about 12 hr to about 36 hr, from about 24 hr to about 48 hr, or any range derivable therein. In certain embodiments, nephrogenic growth factors such as retinoic acid, activin-A, BMP4, and/or BMP7 may be present for incubation for about 24 hr, 36 hr, 48 hr, or 72 hr. In certain embodiments, HDAC inhibitor such as TSA may be present for incubation for about 6 hr, 12 hr, 24 hr, 36 hr, 48 hr, or 72 hr. In certain aspects, epithelial growth factors may be present for incubation for about two days, three days, four days, five days, six days, seven days, eight days, nine days, ten days, or eleven days. In a further aspect, cytokine may be present for incubation for about 24 hr, 36 hr, 48 hr, or 72 hr. The incubation with the nephrogenic growth factor, epithelial growth factor, cytokine, or HDAC inhibitor may be at the same time or sequentially. The time interval between sequential incubation steps may be from about 0 hr to about 24 hr, from about 10 min to about 8 hr, from about 30 min to about 4 hr or any range derivable therein.

Furthermore, an induced renal cell generated according to the methods described above may be provided. Such an induced renal cell may be isolated or in a mixture with the stem and/or progenitor cell.

In certain aspects of the invention, there is provided a method of treating a renal disease or injury comprising the steps of (a) generating an induced renal cell according to any of the methods described above; and (b) administering an effective amount of the induced renal cell to a subject having a renal disease or injury. The renal disease or injury may be acute kidney injury (e.g., acute ischemic/hypoxic kidney injury), chemical or toxin injury, urinary tract obstruction, acute tubular necrosis and apoptosis, glomerular injury and/or inflammation, early dysfunction of kidney transplant, or chronic renal failure. Particularly, the acute kidney failure may be treated with the present invention.

In still further embodiments of the invention, there is provided a culturing system comprising a incubator containing a culture medium, where the culture medium comprises (a) nephrogenic factor selected from the group consisting of a first agent capable of activating a retinoic acid pathway, a second agent capable of activating an activin-A pathway, and a third agent capable of activating a BMP7 pathway; and (b) HDAC inhibitor or HAT agonist. The system may further comprise a lineage undifferentiated hematopoietic stem and/or progenitor cell, and/or an induced renal cell.

In another embodiment, there is provided a method of treating one or more symptoms of renal disease or injury comprising administering to a human subject in need thereof insulin-like growth factor 1 (IGF-1), human growth factor (HGF), vascular endothelial growth factor α (VEGFα) and early growth response 1 (EGR-1). Administering may comprise intravenous, intra-arterial or intraperitoneal administration. The renal disease or injury may be acute kidney injury, chemical or toxin injury, urinary tract obstruction, acute tubular necrosis and apoptosis, glomerular injury and/or inflammation, early dysfunction of kidney transplant, acute ischemic/hypoxic kidney injury or chronic renal failure. The method may further comprise subjecting said subject to dialysis. Administration is via bolus injection or via continuous administration.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating particular embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Source of regenerating cells for renal repair.

FIGS. 2A-B. Effect of histone acetylation and deacetylation. FIG. 2A. The opposing effect of HATs and HDACs. FIG. 2B. Decreased histone deacetylation and increased histone acetylation lead to gene activation. HDAC7 and HDAC9, examples of HDACs. CBP and P/CAF, examples of HATs. TF, transcription factor. AAc, acetyl group.

FIGS. 3A-D. Conversion of BMC into tubular epithelial cells. Y-FISH was followed by immunostaining of nephron segment-specific markers. Y⁺ cells (arrows) were localized to the proximal tubules (pt) stained with LTA (FIG. 3A), thick ascending limbs (tal) stained with anti-NKCC2 (FIG. 3B), distal tubules and connecting tubules (dt) stained with anti-NCX1 (FIG. 3C) and collecting ducts (cd) stained with anti-AQP3 (FIG. 3D). XZ and YZ images indicate the colocalization of Y chromosome with renal markers. Arrowhead indicates a Y⁺ cell in the interstitium in FIG. 3C. Nuclei were counterstained with DAPI.

FIGS. 4A-C. Fusion of bone marrow cells with renal cells. FIG. 4A. Cells containing 3X1Y (arrows) and 2X1Y chromosomes (arrowhead) in the tubules (a), interstitium (b) and the lumen of the tubules (c). Insets are obtained by merging green (X) and red (Y) channels. FIG. 4B. Expression of EYFP by qRT-PCR analysis. mRNA levels were expressed as the % of cre^{ksp}; R26R-EYFP mice that express EYFP constitutively in the kidneys. “1-5” indicate 5 post-ischemic kidneys. FIG. 4C. Expression of EYFP in tubular cells. Laminin (a) stains basement membrane and aPKC (b) stains apical membrane. Z plane images show the colocalization of EYFP with aPKC. Scale bars, 10 μm.

FIGS. 5A-D. Culture and characterization of the cells. FIG. 5A. Lin⁻ cells were exposed to cytokines (1st stage), nephrogenic factors (2nd stage) and epithelial growth factors (3rd stage). Cells were treated with or without TSA (15 nM) for 6 h at the 2nd stage. After the 3rd stage, Lin⁻ cells isolated from cre^{ksp}; R26R-EYFP mice expressed EYFP (green). FIG. 5B. Cell morphology after the 1st and 3rd stages (induction period, 7 day) and after an additional 7 days (total 14 days). Flow cytometry analysis shows the expression of CD45 (FIG. 5C) and CXCR4 (FIG. 5D) in fresh Lin⁻ cells and in cells after the 3^{rd} stage induction.

FIG. 6. Immunostaining shows the expression of CaR in cells after 1st and 3rd stage induction.

FIGS. 7A-D. Induced cells selected renal cell fate. FIG. 7A. Expression of Pax2, Lim1, Gdnf, Six2, Cadherin 6 (Cdh 6), and cadherin 16 (cdh 16) (arrows) in the cells after the 3rd stage, but not after the 1st stage. After the 3rd stage, Lin⁻ cells isolated from cre^{ksp}; R26R-EYFP mice expressed EYFP by RT-PCR (FIG. 7B), immunostaining (arrow in FIG. 7C, right) and FACS analyses (FIG. 7D, right). Cells after the 1st stage have EYFP expression (FIG. 7C, left). No EYFP⁺ cells were detected in fresh Lin⁻ cells (FIG. 7D, left).

FIG. 8. Osr1 expression. Osr1 expression starts after the 2nd stage and increases after the 3rd stage. *p<0.05 compared to 1st stage.

FIG. 9. ChIP assay for acetylation of histone H3 (AcH3) and H4 (AcH4) and tri-methylation (trime-H3) on the promoter regions of cadherin 6 (cdh 6) and cadherin 16 (cdh 16). 1 and 3 denote the cells in the 1st and the 3rd stages. Arrows indicate increased acetylation.

FIG. 10. Expression of HDAC7 and 9 mRNA. Expression of HDAC7 and 9 mRNA by RT-PCR analysis in fresh Lin⁻ cells, and cells after the 1st stage and 3rd stage cultures.

FIG. 11. TSA increases histone H3 and H4 acetylation on renal gene promoters. Equal DNA input was used. IgG was used as a control.

FIG. 12. TSA increases renal cell conversion. Lin⁻ cells were isolated from cre^{ksp}; R26R-EYFP mice. 6.3% cells in vehicle-treated and 37.9% cells in TSA-treated groups are EYFP⁺, indicating a renal cell fate. Wild-type cells (left) were used for gate selection for flow cytometry.

FIG. 13. Induced cells adopt an epithelial phenotype.

FIGS. 14A-B. Induced cells improve renal function and structure. FIG. 14A. BUN levels. FIG. 14B. PAS staining of the kidneys 7 days post IRI.

FIG. 15. 3-D image of tubular integration of induced cell at 3 days post IRI. Y chromosome FISH combined with laminin staining (arrows) shows the presence of Y chromosome signal (Y) in a injured tubule that contains cast.

FIG. 16. Molecular circuitry of hematopoietic-to-renal reprogramming.

FIG. 17. Blocking CaR promotes transmigration of induced cells into post-ischemic kidney where SDF-1 expression is increased.

### DESCRIPTION OF ILLUSTRATIVE EMBODIMENTS

### I. Summary

The instant invention overcomes several major problems with current treatment of renal diseases in providing methods of generating induced renal cells from lineage undifferentiated hematopoietic stem and/or progenitor cells. In certain embodiments, the hematopoietic stem and/or progenitor cells may be incubated with at least one differentiating factor such as a nephrogenic growth factors (e.g., BMP-4, VEGF, bFGF) and an epithelial growth factor. The hematopoietic stem and/or progenitor cells may be further treated with a histone acetylation modulator in order to increase renal conversion efficiency. As stated above, the methods of the present invention provide substantial advantages over previously used methods and culturing systems for the generation of induced renal cells in vitro. Further embodiments and advantages of the invention are described below.

### II. Differentiating Factors

Hematopoietic stem cells (HSCs) have been shown to differentiate into many types of non-hematopoietic cells in vivo and in vitro (Ianus et al., 2003; Jang et al., 2004; Orlic et al., 2001). In the last few years, reports of bone marrow cell conversion into renal cells have emerged (Lin et al., 2003; Lin et al., 2005; Kale et al., 2003; Togel et al., 2005a; Morigi et al., 2004; Togel et al., 2005b; Poulsom et al., 2001; Ito et al., 2001; Gupta et al., 2002; Duffield et al., 2005; Lange et al., 2005; Fang et al., 2005; Sugimoto et al., 2006). The inventors reported freshly isolated HSCs can incorporate into regenerating kidney tubules and bone marrow cells can be converted into tubular epithelial cells, endothelial cells, and glomerular cells in post-ischemic kidneys where the microenvironment is in favor of renal repair (Lin et al., 2005). However, the rate of incorporation was low, and no functional benefit was observed.

To enhance the therapeutic potential of hematopoietic stem/progenitor cells, the inventors have developed a novel approach in which Lin⁻ cells are treated with nephrogenic factors and a histone deacetylase (HDAC) inhibitor or a histone acetyltransferase (HAT) agonist or activator to induce renal differentiation prior to transplantation in certain aspects of the invention. In non-limiting examples, up to 37.9% of treated Lin⁻ cells may express kidney-specific markers and adopt an epithelial-like phenotype under certain defined conditions. To further increase the conversion efficiency, the inventors also incubated or contemplate incubating the hematopoietic stem/progenitor cells with at least a cytokine, a growth factor and/or a mobilizing factor in certain embodiments.

A. Nephrogenic Growth Factors

A combination of nephrogenic growth factors such as retinoic acid, activin-A, and BMP7 has been reported to differentiate embryonic stem (ES) cells into renal epithelial cells that are capable of integrating into a developing kidney (Kim and Dressler, 2005). The inventors found that following treatment of Lin⁻ cells (lineage undifferentiated hematopoietic stem and/or progenitor cells) with a combination of retinoic acid, activin-A, and BMP7, cells may adopt an epithelial phenotype under the induction conditions. However, expression of CD45, CXCR4 and CaR in the majority of induced cells suggests that conversion may be incomplete. It may also suggest that the cells after the induction are heterogenous and the conversion may occur as stochastic events that have been reported in the induction of fibroblasts to iPS cells (Jaenisch and Young, 2008).

Therefore, to overcome these problems, in certain embodiments of the invention, lineage undifferentiated hematopoietic stem and/or progenitor cells (Lin⁻ cell) may be incubated with one or more nephrogenic growth factors in combination with a histone acetylation modulator and/or an epithelial growth factor to induce a renal cell fate. The nephrogenic growth factor used in the present invention is not limited to particular renal differentiation agents. Any nephrogenic factor that may be used to induce a renal fate of the undifferentiated cells is contemplated in the present invention.

In certain embodiments, the nephrogenic factor comprises at least one, two or three of the group consisting of a first agent capable of activating a retinoic acid pathway, a second agent capable of activating an activin-A pathway, and a third agent capable of activating a BMP7 pathway. For example, the first agent may be retinoic acid; the second agent may be activin-A; the third agent may be BMP7.

In particular embodiments, the present invention provides incubation methods in the presence of one or more of activin-A (see, e.g., Hollnagel et al., 1999a; herein incorporated by reference in its entirety), BMP4 (see, e.g., Hollnagel et al., 1999b; Wiles, 1999) each herein incorporated by reference in its entirety), BMP7 (see, e.g., Komaki, et al., 2004; herein incorporated by reference in its entirety) and retinoic acid (see, e.g., Slager et al., 1993; Bain et al., 1995; each herein incorporated by reference in its entirety).

In more particular embodiments, the present invention provides methods and renal cells generated through differentiation of lineage undifferentiated stem or progenitor cells with a nephrogenic cocktail (e.g., activin-A, BMP7, and retinoic acid, or variants thereof).

In some embodiments, stem/progenitor cells are differentiated with a molecule that activates a downstream differentiation factor pathway molecule (e.g., pathway related to retinoic acid, activin-A, and/or BMP7), or derivatives of any of these compounds or similar compounds). In other embodiments, the function of the stem or progenitor cells are modulated with a molecule that activates a downstream growth factor pathway molecule, such as a molecule that activates a pathway related to retinoic acid, activin-A, and/or BMP7.

The term “retinoic acid signaling pathway,” as used in this disclosure, refers to any molecular pathway involving retinoic acid (e.g., any pathway which is influenced by retinoic acid). Such a pathway may be activated by retinoic acid or by homologues or mimics of retinoic acid function (e.g., retinoic acid receptor agonists). Retinoic acid is the oxidized form of Vitamin A.

The term “activin-A signaling pathway,” as used in this disclosure, refers to any molecular pathway involving activin-A (e.g., any pathway which is influenced by activin-A). Such a pathway may be activated by activin-A or by homologues or mimics of activin-A function (e.g., activin-A receptor agonists). Activin is a peptide that enhances FSH synthesis and secretion and participates in the regulation of the menstrual cycle. It performs the opposite function from inhibin. Many other functions have been found to be exerted by activin, including their roles in cell proliferation, differentiation, apoptosis, metabolism, homeostasis, immune response, wound repair, and endocrine function. Like inhibin (and AMH), activin belongs to the TGF-β superfamily. Activin also regulates the morphogenesis of branching organs such as the prostate, lung, and especially kidney. Activin-A increased the expression level of type-I collagen suggesting that activin-A acts as a potent activator of fibroblasts.

The term “BMP7 signaling pathway,” as used in this disclosure, refers to any molecular pathway involving BMP7 (e.g., any pathway which is influenced by BMP7). Such a pathway may be activated by BMP7 or by homologues or mimics of BMP7 function (e.g., BMP7 receptor agonists). Bone morphogenetic protein 7 or BMP7 is a member of the TGF-β superfamily. Like other members of the bone morphogenetic protein family of proteins, it plays a key role in the transformation of mesenchymal cells into bone and cartilage. It is inhibited by noggin and a similar protein, chordin, which are expressed in the Spemann-Mangold Organizer. It is expressed in the brain, kidneys and bladder. BMP7 also has the potential for treatment of chronic kidney disease (Gould et al., 2002; González et al., 2002).

In certain embodiments, individual or a combination of nephrogenic growth factors for inducing renal cells (e.g., retinoic acid, activin-A, and/or BMP7) in the present invention may be used at a concentration of from about 2.5 to about 500 ng/ml, 5 to about 500 ng/ml, from about 10 to about 200 ng/ml, from about 5 to about 100 ng/ml, from about 25 to about 200 ng/ml, from about 25 to about 75 ng/ml, or any range derivable therein. In certain embodiments, retinoic acid, activin-A, BMP4, and/or BMP7 may be used at a concentration of about 2.5, 5, 10, 15, 20, 25, 30, 35, 40, 45, 50, 55, 60, 65, 70, 75, 80, 85, 90, 95 or about 100 ng/ml.

B. Histone Acetylation Regulators

In certain aspects, the stem or progenitor cells may also be treated with a histone acetylation regulator such as a HDAC inhibitor as the inventors found that addition of a HDAC inhibitor may increase renal conversion efficiency at 6-fold by non-limiting examples.

While all cells may carry the same genetic information, distinct cellular phenotypes are largely determined by epigenetic status, including chromatin modification and DNA methylation. Epigenetic state of the cells is not irreversibly fixed and can be changed by nuclear reprogramming. Recent induction of fibroblasts into ES-like cells by defined transcription factors is one of the best examples of cell conversion (Takahashi et al., 2007; Takahashi and Yamanaka, 2006; Wernig et al., 2007; Yu et al., 2007). Mechanisms of cell conversion have begun to unfold and chromatin modifications have been shown to be important in cell conversion.

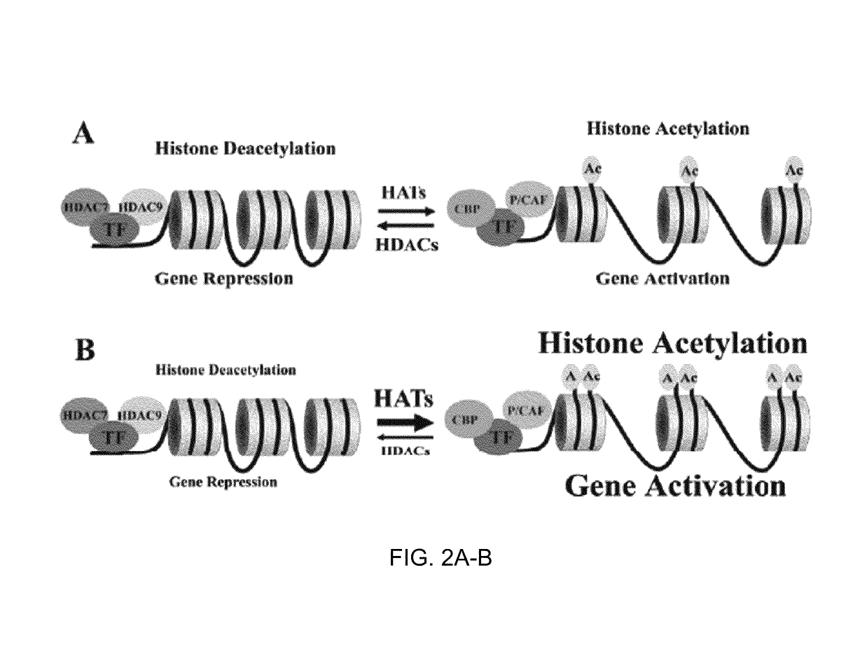

Chromatin is composed of nucleosomes that consist of a histone octamer wrapped inside 146 by of DNA. There are four core histones, H2A, H2B, H3 and H4. Several basic amino acids located at the N-terminus of the histone can be modified by acetylation, methylation, phosphorylation, and ubiquitination. As shown in FIGS. 2A-B, histone acetyltransferases (HATs) acetylate histones and relax the compact structure of nucleosomes, making chromatin more accessible for binding of transcriptional regulators. On the other hand, histone deacetylases (HDACs) reverse this process. Increased histone acetylation is usually associated with gene activation and hypo-acetylated histone is associated with gene repression. HATs and HDACs are recruited to the target genes through association with specific transcription factors that bind to DNA (Jenuwein and Allis, 2001). Transcription coactivators CBP and P/CAF have intrinsic HAT activity and can be recruited to the chromatin remodeling complex to acetylate histones. On the other hand, methylation mediated by DNA methyltransferase can be associated with transcriptional activation or repression depending on the state of methylation.

Chromatin remodeling is critical for epigenetic control of cell type- and stage-specific gene expression. It is involved in stem cell self-renewal, cell fate determination, and differentiation (Akashi et al., 2003; Alexanian, 2007; Cerny and Quesenberry, 2004; Jaenisch and Bird, 2003; Kondo and Raff, 2004; Lee et al., 2006a; Meshorer et al., 2006; Snykers et al., 2007; Tayaramma et al., 2006; Watanabe et al., 2004; Xi and Xie, 2005). Expression of four transcription factors (Oct3/4, Sox2, c-Myc and Klf4) triggers a sequence of stochastic epigenetic events and reprograms fibroblasts into ES-like cells (Wernig et al., 2007; Okita et al., 2007; Maherali et al., 2007). This reprogramming is promoted by methylation of Oct4 promoter and demethylation of Nanog promoter (Wernig et al., 2007). Treatment of neurosphere with inhibitors of HDAC and methyltransferase yields hematopoietic cells that differentiate into multiple types of blood cells in vivo (Schmittwolf et al., 2005). HDAC inhibitor trichostatin A led to differentiation of insulin-producing cells from bone marrow stem cells (Tayaramma et al., 2006). These findings indicate that differentiated cells can undergo chromatin remodeling and nuclear reprogramming to convert to other types of cells, including pluripotent cells, but the differentiation of hematopoietic stem/progenitor cells into renal cells in vitro has not been disclosed until this invention to the inventors' knowledge.

In certain embodiments, the present invention provides for treating, contacting or incubating a Lin⁻ cell with a histone acetylation regulator, for example, a histone deacetylase inhibitor (HDAC) and/or an agent to stimulate histone acetylation such as a histone acetyltransferase (HAT) activator.

A “histone deacetylase inhibitor” is meant to describe any suitable agent that inhibits an enzyme that removes acetyl groups from proteins, in particular histone proteins. Histone deacetylase inhibitor may be small molecule, siRNA, antisense RNA or antibody specific for inhibiting histone deacetylase activity and/or reducing histone deacetylase expression. A “histone acetyltransferase activator” is meant to describe any suitable agent that promotes or stimulates an enzyme that acetylates one or more amino acids on proteins, in particular histone proteins, such as agents that upregulate histone acetyltransferase activity and/or expression level.

Class I HDACs (HDAC1, 2, 3, 8 and 11) and class II HDACs (HDAC4, 5, 7 and 9) contain zinc in the catalytic sites and are inhibited by HDAC inhibitors (HDACi). HDACi such as trichostatin A (TSA) and vorinostat (Zolinza) have been shown to have an anti-cancer effect by inducing apoptosis and terminal differentiation of transformed cells. The effect of HDACi is concentration dependent. Higher concentration often causes apoptosis whereas lower concentration often leads to cell differentiation without apparent cytotoxicity (Carey and La Thangue, 2006). Vorinostat has been approved by U.S. Food and Drug Administration (FDA) in 2006 for treatment of cutaneous T-cell lymphoma.

Despite the ubiquitous distribution of HDACs, HDACi selectively alter a relatively small proportion of expressed genes. TSA alters only 2% of 340 expressed genes in a lymphoid cell line (Van Lint et al., 1996). Microarray studies show alteration of 7-10% genes by HDACi in various cell lines (Gray et al., 2004; Lee et al., 2006b; Mitsiades et al., 2004; Peart et al., 2005). Although the mechanism of selectivity is not well understood, HDCAi may be useful in selective induction of cell differentiation.

Specific non-limiting examples of HDAC inhibitors suitable for use in the methods of the present invention are:
Hydroxamic acid derivatives (e.g., pyroxamide, CBHA, Trichostatin A (TSA), Trichostatin C, hydroxamic acid (SAHA) (approved by the FDA in 2007 for leukemia therapy under the name Vorinostat), Belinostat/PXD101, LBH589, ITF2357, suberoylanilide Salicylihydroxamic Acid (SBHA), Azelaic Bishydroxamic Acid (ABHA), Azelaic-1-Hydroxamate-9-Anilid-e (AAHA), 6-(3-Chlorophenylureido) carpoic Hydroxamic Acid (3Cl-UCHA), Oxamflatin, A-161906, Scriptaid, PXD-101, CHAP, MW2796, and MW2996);
Cyclic peptides (e.g., trapoxin A, FR901228 (FK 228, depsipeptide), FR225497, Apicidin, CHAP, HC-Toxin, WF27082, and chlamydocin);
Short chain fatty acids (SCFAs) or aliphatic acids (e.g., sodium butyrate, butyrate, isovalerate, valerate, AN-9, 4 phenylbutyrate (4-PBA), phenylbutyrate (PB), propionate, butyramide, isobutyramide, phenylacetate, 3-bromopropionate, tributyrin, valproic acid and valproate);
Benzamide derivatives (e.g., MS-275, CI-994, MS-27-275 (MS-275), a 3′-amino derivative of MS-27-275, and MGCD0103);
Electrophilic ketone derivatives (e.g., a trifluoromethyl ketone and an α-keto amide such as an N-methyl-α-ketoamide); and Depudecin.

In certain embodiments, downregulation of HDAC activity in Lin⁻ cells could use anti-sense mRNA or siRNA directed to one or more specific HDACs such as HDAC4, HDAC7 and HDAC9.

Histone acetyltransferases (HAT) are enzymes that acetylate conserved lysine amino acids on histone proteins by transferring an acetyl group from acetyl CoA to lysine to form ε-N-acetyl lysine. The inventors have contemplated to use a HAT activator known in the art, for example, overexpression of an enzyme with HAC activity such as CBP and/or P/CAF in Lin⁻ cells to increase renal conversion.

C. Growth Factors

In certain embodiments of the invention, the hematopoietic stem and/or progenitor cells may be incubated with one or more growth factor, more particularly epithelial growth factor, to promote epithelial growth. Suitable growth factors include, but are not limited to, epidermal growth factor (EGF), hepatocyte growth factor/scatter factor (HGF/SF), transforming growth factor β (TGF β), any type of fibroblast growth factor (exemplified by FGF-4, FGF-8, and basic fibroblast growth factor (bFGF or FGF-2)), platelet-derived growth factor (PDGF), insulin-like growth factor (IGF-1 and others), high concentrations of insulin, sonic hedgehog, members of the neurotrophin family (such as nerve growth factor, neurotrophin 3, brain-derived neurotrophic factor), additional bone morphogenic proteins, and ligands to receptors that complex with gp 130 (such as LIF, CNTF, and IL-6). Also suitable are alternative ligands and antibodies that bind to the respective cell-surface receptors for the aforementioned factors.

Particularly, a cocktail or a culture medium containing a plurality of epithelial growth factor may be used, which may comprise 2, 3, 4, or more of the agents listed above or in the examples below. The present invention may also be practiced with growth factor variants, such as deletion mutants, sequence change variants, truncated versions of particular differentiation factor, small molecule mimetics, etc.

In certain embodiments, the present invention may use at least an epithelial growth factor selected from the group consisting of EGF, IGF-1 and HGF, their functional equivalents and combination thereof to contact or incubate the stem and/or progenitor cells. In certain embodiments, individual or a combination of growth factors for inducing renal cells (e.g., EGF, IGF-1 and HGF) in the present invention may be used at a concentration of from about 2.5 to about 500 ng/ml, 5 to about 500 ng/ml, from about 10 to about 200 ng/ml, from about 5 to about 100 ng/ml, from about 25 to about 200 ng/ml, from about 25 to about 75 ng/ml, or any range derivable therein. In certain embodiments, EGF, IGF-1 and HGF may be used at a concentration of about 2.5, 5, 10, 15, 20, 25, 30, 35, 40, 45, 50, 55, 60, 65, 70, 75, 80, 85, 90, 95 or about 100 ng/ml.

Epidermal growth factor or EGF is a growth factor that plays an important role in the regulation of cell growth, proliferation, and differentiation by binding to its receptor EGFR. EGF acts by binding with high affinity to epidermal growth factor receptor (EGFR) on the cell surface and stimulating the intrinsic protein-tyrosine kinase activity of the receptor (see the second diagram). The tyrosine kinase activity, in turn, initiates a signal transduction cascade that results in a variety of biochemical changes within the cell—a rise in intracellular calcium levels, increased glycolysis and protein synthesis, and increases in the expression of certain genes including the gene for EGFR—that ultimately lead to DNA synthesis and cell proliferation.

EGF is the founding member of the EGF-family of proteins. Members of this protein family have highly similar structural and functional characteristics. Besides EGF itself other family members include (Dreux et al., 2006): Heparin-binding EGF-like growth factor (HB-EGF), transforming growth factor-α (TGF-α), Amphiregulin (AR), Epiregulin (EPR), Epigen Betacellulin (BTC), neuregulin-1 (NRG1), neuregulin-2 (NRG2), neuregulin-3 (NRG3), neureguline-4 (NRG4). All family members contain one or more repeats of the conserved amino acid sequence: CX7CX4-5CX10-13CXCX8GXRC, where X represents any amino acid. This sequence contains 6 cysteine residues that form three intramolecular disulfide bonds. Disulfide bond formation generates three structural loops that are essential for high-affinity binding between members of the EGF-family and their cell-surface receptors (Harris et al., 2003).

The insulin-like growth factors (IGFs) are polypeptides with high sequence similarity to insulin. IGFs are part of a complex system that cells use to communicate with their physiologic environment. This complex system (often referred to as the IGF “axis”) consists of two cell-surface receptors (IGF1R and IGF2R), two ligands (IGF-1 and IGF-2), a family of six high-affinity IGF binding proteins (IGFBP 1-6), as well as associated IGFBP degrading enzymes, referred to collectively as proteases. Insulin-like growth factor 1 (IGF-1) is mainly secreted by the liver as a result of stimulation by growth hormone (GH). IGF-1 is important for both the regulation of normal physiology, as well as a number of pathological states, including cancer. The IGF axis has been shown to play roles in the promotion of cell proliferation and the inhibition of cell death (apoptosis). Insulin-like growth factor 2 (IGF-2) is thought to be a primary growth factor required for early development while IGF-1 expression is required for achieving maximal growth.

Hepatocyte growth factor/scatter factor (HGF/SF) is a paracrine cellular growth, motility and morphogenic factor by activating a tyrosine kinase signaling cascade after binding to the proto-oncogenic c-Met receptor. It is secreted by mesenchymal cells and targets and acts primarily upon epithelial cells and endothelial cells, but also acts on hematopoietic progenitor cells. Its ability to stimulate mitogenesis, cell motility, and matrix invasion gives it a central role in angiogenesis, tumorogenesis, and tissue regeneration. It has been shown to have a major role in embryonic organ development, in adult organ regeneration and in wound healing (Gallagher and Lyon, 2000). It is secreted as a single inactive polypeptide and is cleaved by serine proteases into a 69-kDa α-chain and 34-kDa β-chain. A disulfide bond between the α and β chains produces the active, heterodimeric molecule. The protein belongs to the plasminogen subfamily of S1 peptidases but has no detectable protease activity. Alternative splicing of this gene produces multiple transcript variants encoding different isoforms, which could also be used in the present invention.

D. Cytokines

In certain embodiments of the invention, the hematopoietic stem and/or progenitor cells may be incubated with one or more cytokines to induce the cell into cell cycle.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α. and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, KIT ligand (stem cell factor) or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Particularly, the cytokine used in the present invention may be an agent capable of activating stem cell factor pathway, an agent capable of activating IL-3 pathway and/or an agent capable of activating IL-6 pathway.

Stem cell factor (SCF), otherwise known as KIT ligand or Steel factor, is a cytokine which binds CD117 (c-Kit). SCF is also known as “steel factor” or “c-kit ligand.” SCF exists in two forms, cell surface bound SCF and soluble (or free) SCF. Soluble SCF is produced by the cleavage of surface bound SCF by metalloproteases.

SCF is a growth factor important for the survival, proliferation, and differentiation of hematopoietic stem cells and other hematopoietic progenitor cells. One of its roles is to change the BFU-E (burst-forming unit-erythroid) cells, which are the earliest erythrocyte precursors in the erythrocytic series, into the CFU-E (colony-forming unit-erythroid). SCF, along with bFGF (basic fibroblast growth factor) and LIF (lymphocyte inhibitory factor), prevents spontaneous differentiation of primitive embryonic stem cells in cell culture. Ancestim is a recombinant form of SCF.

Interleukin-3 (IL-3) (colony-stimulating factor, multiple), also known as IL-3, is an interleukin, a type of biological signal (cytokine) that can improve the body's natural response to disease as part of the immune system. It acts by binding to the interleukin-3 receptor. IL-3 stimulates the proliferation of hematopoietic pluripotent progenitor cells. It is secreted by activated T cells to support growth and differentiation of T cells from the bone marrow in an immune response. The human IL-3 gene encodes a protein 152 amino acids long, and the naturally occurring IL-3 is glycosylated. The human IL-3 gene is located on chromosome 5, only 9 kilobases from the GM-CSF gene, and its function is quite similar to GM-CSF.

Interleukin-6 (IL-6) is an interleukin that acts as both a pro-inflammatory and anti-inflammatory cytokine. It is secreted by T cells and macrophages to stimulate immune response to trauma, especially burns or other tissue damage leading to inflammation. IL-6 is also a “myokine,” a cytokine produced from muscle, and is elevated in response to muscle contraction. It is significantly elevated with exercise, and precedes the appearance of other cytokines in the circulation. During exercise, it is thought to act in a hormone-like manner to mobilize extracellular substrates and/or augment substrate delivery (Petersen, 2005). Additionally, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-α and IL-1, and activation of IL-1ra and IL-10.

E. Mobilizing Factors

One of the key features of HSC is their ability to migrate in a site-specific fashion. Under normal and transplant conditions, HSC prefer to home to the bone marrow stem cell niches. Upon stimulation, HSC can be mobilized into the peripheral organs, such as ischemic/hypoxic tissues (Kaplan et al., 2007). Homing and mobilization share similar molecular mechanisms (Chute, 2006; Kucia et al., 2005; Lapidot et al., 2005). Among cell adhesion molecules that are involved in homing and mobilization, the pairs of stromal derived factor-1 (SDF-1)/its receptor CXCR4 (Kucia et al., 2005) and calcium sensing receptor (CaR)/Ca²⁺ are the major players.

SDF-1 belongs to the chemokine family and is expressed by a wide variety of cells including bone marrow stromal cells and endothelial cells. Binding of SDF-1 with its receptor CXCR4, a G-protein coupled glycoprotein expressed on HSC and progenitor cells, elicits cell adhesion and transmigration. Blockade of SDF-1/CXCR4 dramatically reduces HSC homing (Bonig et al., 2006). In the normal kidney, SDF-1 is expressed in distal tubules. Proximal tubules also express low levels of SDF-1. SDF-1 expression is up-regulated 2-24 hr following ischemic injury. Conversely, SDF-1 protein levels decrease in the bone marrow after renal IRI. The changes of SDF-1 gradient favor the migration of HSC and progenitor cells into the circulation and enter the kidney (Togel et al., 2005b).

In certain embodiments of the present invention, the Lin⁻ cells or the induced renal cells may be treated with a mobilization factor by decreasing their homing to bone marrow and/or increasing the transmigration to renal tissues or tissues adjacent to kidney. The cells may be mobilized with granulocyte-colony stimulating factor (G-CSF), and/or stem cell factor (SCF), granulocyte-macrophage colony-stimulating factor (GM-CSF), macrophage colony-stimulating factor (M-CSF), interleukin-1 (IL-1), interleukin-2 (IL-2), interleukin-3 (IL-3), interleukin-4 (IL-4), interleukin-6 (IL-6), interleukin-7 (IL-7), interleukin-8 (IL-8), interleukin-11 (IL-11), interleukin-12 (IL-12), Flt3-L, TPO and EPO, or any stem cell mobilization factor known to one skilled in the art. Since injured organs, unlike intact organs, generate homing signals via activation of their cognate receptors that attract stem cells, this form of therapy is optimally focused on the protection and repair of a damaged kidney or other organ. A preferred stem cell mobilization factor does not simultaneously increase peripheral neutrophil numbers, causing granulocytosis, when the stem cells are mobilized for treatment of a patient having kidney or other organ injury.

Particularly, the inventors contemplate using CaR blockers such as a CaR antibody, a CaR siRNA or CaR small molecule inhibitor to promote transmigration of induced renal cells into the kidney.

CaR is a G protein-coupled cell surface receptor expressed on HSC and some lineage differentiated white blood cells, parathyroid gland and kidney epithelial cells (Riccardi et al., 1996; Kos et al., 2003; House et al., 1997; Olszak et al., 2000). In response to high Ca²⁺ concentration in the endosteum of the bone marrow (as high as 40 mM, 20 times of serum concentration), HSC migrate and engraft to the endosteal niche. HSC CaR deficient mice show a profound defect in, homing to the bone marrow (Adams et al., 2006). The effect of Ca²⁺ and CaR on HSC engraftment provides a potential target for decreasing bone marrow homing and increasing transmigration into post ischemic kidneys.

### III. Stem and/or Progenitor Cells

Lineage undifferentiated hematopoiteic stem cells or hematopoiteic progenitor cells (Lin⁻ cells) may be used in the present invention to induce renal cell differentiation. The cells can be characterized by the absence of lineage-specific markers for white blood cells, red blood cells, and platelets. These cells may include hematopoiteic stem cells alone, lineage undifferentiated hematopoiteic progenitor cells (such as early multipotent progenitors) alone or the combination of the two cell populations. The inventors have made major progress to increase the efficiency of conversion of these Lin⁻ cells into renal cells for renal therapy. The present invention also contemplates the use of non-hematopoietic stem cells such as embryonic stem cell or induced pluripotent stem cells because of their pluripotency.

A. Hematopoietic Stem and/or Progenitor Cell Overview

A “lin-negative” (Lin⁻) cell is known to one of skill in the art as being a cell that does not express antigens characteristic of specific blood cell lineages and thus is more primitive, multipotent. Lin⁻ cells, including hematopoietic stem cells and/or lineage undifferentiated progenitor cells, which are negative for the markers that are used for detection of lineage commitment, are used in the present invention. Any method known to one of skill in the art may be used to enrich the population of multipotent stem cells from the whole population of bone marrow cells, and, if necessary, cryopreserve them until needed for treatment or incubation.

Hematopoietic stem cells (HSCs) are stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). The discovery of HSC by Till and McCulloch (1961) led to the definition of all stem cell characteristics: self-renewal and lineage differentiation. Lin⁻Sca-1⁺c-kit⁺ HSC were isolated from mouse bone marrow by fluorescence-activated cell sorter (FACS) and were shown to provide long-term reconstitution of all types of blood cells in the transplanted mice (Spangrude et al., 1988; Ikuta and Weissman, 1992). It is generally agreed that the earliest hematopoiesis occurs at the yolk sac and aorta-gonad-mesonephros (AGM) region (Medvinsky and Dzierzak, 1996). The common mesodermal origin suggests that HSC and embryonic kidney may share similar genetic programs. The HSC may be CD 34⁺ or CD34⁻.

In one embodiment of the present invention, the multipotent stem cell population may be derived from HSCs. The HSCs are derived from the bone marrow or peripheral blood, preferably the bone marrow. The HSCs are isolated from a healthy and compatible donor or the patients themselves (autologous) by techniques commonly known in the art. The HSC population may be enriched for pluripotent HSC using fluorescence activated cell sorting (FACS) or other methods. The pluripotent HSC may be enriched by FACS by selecting for “c-kit” positive, “sca-1” positive and “lin-negative” cells because “c-kit” and “sca-1” are known to one of skill in the art as being receptors known to be on the surface of stem cells.

HSCs may be found in the bone marrow of adults, which includes femurs, hip, ribs, sternum, and other bones. Cells can be obtained directly by removal from the hip using a needle and syringe, or from the blood following pre-treatment with cytokines, such as G-CSF (granulocyte colony-stimulating factors), that induce cells to be released from the bone marrow compartment. Other sources for clinical and scientific use include umbilical cord blood, placenta, mobilized peripheral blood. For experimental purposes, fetal liver, fetal spleen, and AGM (Aorta-gonad-mesonephros) of animals are also useful sources of HSCs.

B. Hematopoietic Stem/Progenitor Cell Characteristics

HSCs are defined by their ability to form multiple cell types (multipotency) and their ability to self-renew.

The multipotency of HSCs is evidenced by their capacity to differentiate into multiple lineages within the blood and immune system, as well as cells of non-hematopoietic tissues, such as hepatocytes, cardiac myocytes, gastrointestinal epithelial cells, and vascular endothelial cells. The discovery that adult HSC can cross lineage boundaries to become cells of other tissues has challenged the traditional view that somatic stem cells are lineage-restricted and organ-specific. One possibility is that HSC retain developmental plasticity and can be reprogrammed to express genes that are required to differentiate into the cells of the organs into which they are introduced. Another distinguishing feature of HSC is their ready availability from bone marrow, cord blood, and mobilized peripheral blood. This property makes HSC potentially useful for cell replacement therapy in regenerative medicine.

It is known that a small number of HSCs can expand to generate a very large number of progeny HSCs. This phenomenon is used in bone marrow transplant when a small number of HSCs reconstitute the hematopoietic system. This indicates that, at least during bone marrow transplant, symmetrical cell divisions that give two progeny HSCs must occur, as expansion in HSC numbers seen during bone marrow transplant cannot occur in any other way.

Stem cell self-renewal is thought to occur in the stem cell niche in the bone marrow, and it is reasonable to assume that key signals present in this niche will be important in self-renewal. There is much interest in the environmental and molecular requirements for HSC self-renewal, as understanding the ability of HSC to reprogram or differentiate will eventually allow the generation of induce renal cells in vitro that can be used therapeutically.

C. Hematopoietic Stem/Progenitor Cell Isolation

Lin⁻ cells, including hematopoietic stem cells and/or lineage undifferentiated progenitor cells, are negative for the markers that are used for detection of lineage commitment; during their purification by FACS, up to 14 different mature blood-lineage marker (e.g., CD13 and CD33 for myeloid, CD71 for erythroid, CD19 for B cells, CD61 for megakaryocytic, etc., for humans, B220 for B cells, Mac-1 (CD11b/CD18) for monocytes, Gr-1 for Granulocytes, Ter119 for erythroid cells, IL7Ra, CD3, CD4, CD5, CD8 for T cells, etc. for mice) antibodies are used as a mixture to deplete the Lin⁺ cells or late multipotent progenitors (MPPs).

In reference to phenotype, hematopoeitic stem cells are also identified by their small size, lack of lineage (lin) markers, low staining (side population) with vital dyes such as rhodamine 123 (rhodamineDULL, also called rholo) or Hoechst 33342, and presence of various antigenic markers on their surface, many of which belong to the cluster of differentiation series, like CD34, CD38, CD90, CD133, CD105, CD45 and also c-kit—the receptor for stem cell factor.

Although they are rare populations representing 0.005% to 0.01% of total bone marrow cells, HSC can be isolated by fluorescence-activated cell sorting (FACS) based on their expression of cell surface markers and their ability to efflux mitochondrial dyes (Spangrude et al., 1988; Wolf et al., 1993), such as rhodamine-123 (Rh). The inventors previously reported that primitive Rh^{lo} Lin⁻Sca-1⁺ ckit⁺ cells supported hematopoiesis for up to 6 months in lethally irradiated mice and contributed to blood formation in secondary recipients (Park et al., 2002). Using bioinformatics and microarray analysis, the inventors also showed that these primitive cells exhibit distinct gene expression patterns compared with less primitive bone marrow cells, suggesting that differentially expressed genes may govern the phenotype and function of hematopoietic cells (Park et al., 2002; Akashi et al., 2002).

D. Cell Homing and Mobilization

Stem cell niche refers to a microenvironment where HSC reside and maintain the potential of self-renewal (Schofield, 1978). Endosteal space of the bone marrow has been identified as HSC niche and the osteoblasts lining the inner bone surface are the important niche cells controlling the number of HSC (Zhang et al., 2003; Calvi et al., 2003). More recently, endothelia of the sinusoidal vessels of the bone marrow were found to be the vascular niche for HSC (Kopp et al., 2005; Kiel et al., 2005). One of the key features of HSC is their ability to migrate in a site-specific fashion. Under normal and transplant conditions, HSC prefer to home to the bone marrow stem cell niches. Upon stimulation, HSC can be mobilized into the peripheral organs, such as ischemic/hypoxic tissues (Kaplan et al., 2007). Homing and mobilization share similar molecular mechanisms (Chute, 2006; Kucia et al., 2005; Lapidot et al., 2005). Among cell adhesion molecules that are involved in homing and mobilization, the pairs of stromal derived factor-1 (SDF-1)/its receptor CXCR4 (Kucia et al., 2005) and calcium sensing receptor (CaR)/Ca²⁺ are the major players. As disclosed above, the present invention may use mobilizing factors such as a CaR blocker to promote induced renal cells to transmigrate to kidney.

E. Umbilical Cord Stem Cells.

Human umbilical cord blood contains a significantly higher concentration of hematopoietic stem and progenitor cells and can be easily obtained with minimal ethical concerns. Furthermore, once cord blood banking becomes a standard of practice, autologous cells can be used without the need of immunosuppression. The present invention includes using umbilical cord stem cells to induce renal cell conversion. The easy access of human umbilical cord stem cells and minimal ethical concerns renders these stem cells an ideal cellular source to treat kidney disease. Hematopoietic stem cells and progenitor cells in the human umbilical cord blood can be isolated, for example, by immunobead methods to select CD34⁺ cells.

F. Embryonic Stem Cells

In certain embodiments, the present invention may utilize embryonic stem cells. Embryonic stem cells are pluripotent cells derived from the inner cell mass of pre-implantation embryos (Evans et al., 1981; herein incorporated by reference in its entirety). Embryonic stem cells can differentiate into any cell type in vivo (Bradley et al., 1984; Nagy et al., 1990; each herein incorporated by reference in their entireties) and into a more limited variety of cells in vitro (Doetschman et al., 1985; Wobus et al., 1988; Robbins et al., 1990; Schmitt et al., 1991; each herein incorporated by reference in their entireties).

Mouse embryonic stem cells, however, are more difficult to maintain in the laboratory and require the addition of a differentiation inhibitory factor (commonly referred to as leukemia inhibitory factor (or LIF) in the culture medium to prevent spontaneous differentiation (Williams et al., 1988; Smith et al., 1988; Gearing et al., 1989; Pease et al., 1990a; each herein incorporated by reference in their entireties). LIF is a secreted protein and can be provided by maintaining embryonic stem cells on a feeder layer of cells that produce LIF (Evans et al., 1981; Robertson, 1987; herein incorporated by reference in its entirety) or by the addition of purified LIF (Williams et al., 1988; Smith et al., 1988; Gearing et al., 1989; Pease et al., 1990b; each herein incorporated by reference in their entireties) to the medium in the absence of feeder layers.

Differentiation of embryonic stem cells into a heterogeneous mixture of cells occurs spontaneously if LIF is removed, and can be induced further by manipulation of culture conditions (Doetschman et al., 1985; Wobus et al., 1988; Robbins et al., 1990; Schmitt et al., 1991; Wiles et al., 1991; Gutierrez-Ramos et al., 1992; each herein incorporated by reference in their entireties). Embryonic stem cell differentiation can be variable between different established embryonic stem cell lines and even between laboratories using the same embryonic stem cell lines.

Embryonic stem cells can be isolated, for example, from blastocysts of members of the primate species (see, e.g., Thomson et al., 1995; herein incorporated by reference in its entirety). Human embryonic stem (hES) cells can be prepared from human blastocyst cells using the techniques described by Thomson et al. (see, e.g., U.S. Pat. No. 5,843,780; 1998a; 1998b; each herein incorporated by reference in their entireties) and Reubinoff et al. (2000), which also is herein incorporated by reference in its entirety.

G. Induced Pluripotent Stem Cells

In the present invention, iPSCs may also be used to differentiate into renal cells for renal therapy. Induced pluripotent stem cells, commonly abbreviated as iPS cells or iPSCs, are a type of pluripotent stem cell artificially derived from a non-pluripotent cell, typically an adult somatic cell, by inducing a “forced” expression of certain genes. iPSCs were capable of differentiation in a fashion similar to ESCs into fully differentiated tissues.

iPSCs are believed to resemble natural pluripotent stem cells, such as embryonic stem cells in many respects, such as the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed.

iPSCs were first produced in 2006 from mouse cells (Takahashi and Yamanaka, 2006) and in 2007 from human cells (Yu et al., 2007). This has been cited as an important advancement in stem cell research, as it may allow researchers to obtain pluripotent stem cells, which are important in research and potentially have therapeutic uses, without the controversial use of embryos.

### IV. Renal Conditions

The conditions identified for treatment with induced renal cells in the present invention with induced renal cells, including, but not limited to, acute kidney injury (AKI) or acute kidney failure (ARF), acute tubular necrosis and apoptosis, glomerular injury and/or inflammation, diabetic nephropathy, dysfunction of kidney transplant, chronic renal failure, multi-organ failure, and kidney dysfunction. Descriptions of these conditions may be found in medical texts, such as The Kidney, 2001, which is incorporated herein in its entirety by reference. The treatment methods may also be used for related diseases or conditions, such as cardiovascular diseases.

A. Acute Kidney Injury (AKI)

Acute kidney injury (AKI), also known as acute renal failure (ARF) or acute kidney failure, is a rapid loss of renal function due to damage to the kidneys, resulting in retention of nitrogenous (urea and creatinine) and non-nitrogenous waste products that are normally excreted by the kidney. AKI may be defined as an acute deterioration in renal excretory function within hours or days. AKI may include AKI of native kidneys, AKI of native kidneys in multi-organ failure, and AKI in transplanted kidneys. In severe AKI, the urine output is absent or very low.

As a consequence of this abrupt loss in function, azotemia develops, defined as a rise of serum creatinine levels and blood urea nitrogen levels. Serum creatinine and blood urea nitrogen levels are measured. Uremia can occur due to the parallel accumulation of uremic toxins in the blood. The accumulation of uremic toxins causes bleeding from the intestines, neurological manifestations most seriously affecting the brain, leading, unless treated, to coma, seizures and death. In adults, a normal serum creatinine level is ˜1.0 mg/dL, a normal blood urea nitrogen level is ˜20 mg/dL. A normal serum creatinine level in children is age-dependent and is usually less than 1.0 mg/dL.

Depending on the severity and duration of the renal dysfunction, this accumulation is accompanied by metabolic disturbances, such as metabolic acidosis (acidification of the blood) and hyperkalemia (elevated potassium levels), changes in body fluid balance, and effects on many other organ systems. For example, acid (hydrogen ions) and potassium levels rise rapidly and dangerously, resulting in cardiac arrhythmias and possible cardiac standstill and death. If fluid intake continues in the absence of urine output, the patient becomes fluid overloaded, resulting in a congested circulation, pulmonary edema and low blood oxygenation, thereby also threatening the patient's life. One of skill in the art interprets these physical and laboratory abnormalities, and bases the needed therapy on these findings. Renal failure can also be characterized by oliguria or anuria (decrease or cessation of urine production), although nonoliguric AKI may occur. It is a serious disease and treated as a medical emergency.

The most common cause of AKI is an ischemic insult of the kidney resulting in injury of renal tubular and vascular endothelial cells. The principal etiologies for this ischemic form of AKI include intravascular volume contraction, resulting from bleeding, thrombotic events, shock, sepsis, major cardiovascular surgery, arterial stenoses, and others. Nephrotoxic forms of AKI can be caused by radiocontrast agents, significant numbers of frequently used medications such as chemotherapeutic drugs, antibiotics and certain immunosuppressants such as cyclosporine. Patients most at risk for all forms of ARF include diabetics, those with underlying kidney, liver, cardiovascular disease, the elderly, recipients of a bone marrow transplant, and those with cancer or other debilitating disorders.

Both ischemic and nephrotoxic forms of AKI result in dysfunction and death of renal tubular and microvascular endothelial cells. Sublethally injured tubular cells dedifferentiate, lose their polarity and express vimentin, a mesenchymal cell marker, and Pax-2, a transcription factor that is normally only expressed in the process of mesenchymal-epithelial transdifferentiation in the embryonic kidney. Injured endothelial cells also exhibit characteristic changes.

Another acute form of renal failure or injury, transplant-associated acute renal failure (TA-ARF), also termed early graft dysfunction (EGD), commonly develops upon kidney transplantation, mainly in patients receiving transplants from cadaveric donors, although TA-ARF may also occur in patients receiving a living related donor kidney. Up to 50% of currently performed kidney transplants utilize cadaveric donors. Kidney recipients who develop significant TA-ARF require treatment with hemodialysis until graft function recovers. The risk of TA-ARF is increased with elderly donors and recipients, marginal graft quality, significant comorbidities and prior transplants in the recipient, and an extended period of time between harvest of the donor kidney from a cadaveric donor and its implantation into the recipient, known as “cold ischemia time.” Early graft dysfunction or TA-ARF has serious long-term consequences, including accelerated graft loss due to progressive, irreversible loss in kidney function that is initiated by TA-ARF, and an increased incidence of acute rejection episodes leading to premature loss of the kidney graft. Therefore, a great need exists to provide a treatment for early graft dysfunction due to TA-ARF or EGD.

The kidney, even after severe acute insults, has the remarkable capacity of self-regeneration and consequent re-establishment of renal structure and function to a certain degree. It is thought that the regeneration of injured nephron segments is the result of dedifferentiation, migration, proliferation and redifferentation of surviving tubular and endothelial cells.

However, the self-regeneration capacity of the surviving tubular and vascular endothelial cells may be exceeded in severe AKI. Patients with isolated AKI from any cause, e.g., AKI that occurs without multi-organ failure, continue to have a mortality in excess of 50%. This dismal prognosis has not improved despite intensive care support, hemodialysis, and the recent use of atrial natriuretic peptide, Insulin-like Growth Factor-I (IGF-I), more biocompatible dialysis membranes, continuous hemodialysis, and other interventions.

The mortality of AKI in humans remains between 50-80% (Schrier et al., 2004; Liano and Pascual, 1998). Moreover, the incidence of AKI is increasing (Xue et al., 2006). Renal recovery from AKI is incomplete. An urgent need exists to enhance the kidney's self-defense and autoregenerative capacity after severe injury. The present invention based on stem cells offer the potential for treatment of AKI.

B. Acute Tubular Necrosis

Acute tubular necrosis or (ATN) is a medical condition involving the death of tubular cells that form the tubule that transports urine to the ureters while reabsorbing 99% of the water (and highly concentrating the salts and metabolic byproducts). Tubular cells continually replace themselves and if the cause of ATN is removed then recovery is likely. ATN presents with acute renal failure and is one of the most common causes of ARF. The presence of “muddy brown casts” of epithelial cells found in the urine during urinalysis is pathognomonic for ATN.

It may be classified as either toxic or ischemic. Toxic ATN occurs when the tubular cells are exposed to a toxic substance (nephrotoxic ATN). Ischemic ATN occurs when the tubular cells do not get enough oxygen, a condition they are highly sensitive to due to their very high metabolism.

Toxic ATN can be caused by free hemoglobin or myoglobin, by medication such as antibiotics and cytostatic drugs, or by intoxication (ethylene glycol, “anti-freeze”). Toxic ATN is characterized by proximal tubular epithelium necrosis (no nuclei, intense eosinophilic homogeneous cytoplasm, but preserved shape) due to a toxic substance (poisons, organic solvents, drugs, heavy metals). Necrotic cells fall into the tubule lumen, obliterating it, and determining acute renal failure. Basement membrane is intact, so the tubular epithelium regeneration is possible. Glomeruli are usually not affected to a significant degree.

Ischemic ATN can be caused when the kidneys are not sufficiently perfused for a long period of time (i.e., renal artery stenosis) or during shock. Hypoperfusion can also be caused by embolism of the renal arteries. Ischemic ATN specifically causes skip lesions through the tubules.

C. Chronic Renal Failure

Chronic renal failure (CRF) or Chronic Kidney Disease (CKD) is the progressive loss of nephrons and consequent loss of renal function, resulting in End Stage Renal Disease (ESRD), at which time patient survival depends on dialysis support or kidney transplantation. The progressive loss of nephrons, i.e., glomeruli, tubuli and microvasculature, appears to result from self-perpetuating fibrotic, inflammatory and sclerosing processes, most prominently manifested in the glomeruli and renal interstitium. The loss of nephrons is most commonly initiated by diabetic nephropathy, glomerulonephritides, many proteinuric disorders, hypertension, vasculitic, inflammatory and other injuries to the kidney.

Currently available forms of therapy, such as the administration of angiotensin converting enzyme inhibitors, angiotensin receptor blockers, other anti-hypertensive and anti-inflammatory drugs such as steroids, cyclosporine and others, lipid lowering agents, omega-3 fatty acids, a low protein diet, and optimal weight, blood pressure and blood sugar control, particularly in diabetics, can significantly slow and occasionally arrest the chronic loss of kidney function in the above conditions. The development of ESRD can be prevented in some compliant patients and delayed others.

Despite these successes, the annual growth of patient numbers with ESRD, requiring chronic dialysis or transplantation, remains at 6%, representing a continuously growing medical and financial burden. There exists an urgent need for the development of new interventions for the effective treatment of CRF or CKD and thereby ESRD, to treat patients who fail to respond to conventional therapy, i.e., whose renal function continues to deteriorate. In certain embodiments of the invention, induced renal cell treatment will be provided to arrest/reverse the fibrotic processes in the kidney.

D. Multi-Organ Failure

Multi-organ failure (MOF) remains a major unresolved medical problem. MOF develops in the most severely ill patients who have sepsis, particularly when the latter develops after major surgery or trauma. It occurs also with greater frequency and severity in elderly patients, those with diabetes mellitus, underlying cardiovascular disease and impaired immune defenses. MOF is characterized by shock, acute renal failure (ARF), leaky cell membranes, dysfunction of lungs, liver, heart, blood vessels and other organs. Mortality due to MOF approaches 100% despite the utilization of the most aggressive forms of therapy, including intubation and ventilatory support, administration of vasopressors and antibiotics, steroids, hemodialysis and parenteral nutrition. Many of these patients have serious impairment of the healing of surgical or trauma wound, and, when infected, these wounds further contribute to recurrent infections, morbidity and death. The induced renal cell-based therapy disclosed in the present invention, may be combined with other organ transplantation or therapeutic methods, to treat or alleviate multi-organ failure.

E. Renal Repair

Therapies that are currently utilized in the treatment of AKI or ARF, the treatment of established ARF of native kidneys per se or as part of MOF, and ARF of the transplanted kidney, and organ failure in general have not succeeded to significantly improve morbidity and mortality in this large group of patients. Consequently, there exists an urgent need for the improved treatment of MOF, renal dysfunction, and renal failure.

Very promising pre-clinical studies in animals and a few early phase clinical trials administer bone marrow-derived hematopoietic stem cells for the repair or protection of one specific organ such as the heart, small blood vessels, brain, spinal cord, liver and others. These treatments have generally used only bone-marrow stem cells, hematopoietic (HSC) and/or mesenchymal stem cells (MSC), and obtained results are very encouraging in experimental stroke, spinal cord injury, and myocardial infarction.

Mouse ischemia-reperfusion injury (IRI) is a well-established model to study human acute kidney injury (AKI). The main pathogenesis of IRI includes acute tubular necrosis and apoptosis, glomerular injury and inflammation (Schrier et al., 2004; Bonventre, 1993). Injury results in decreased renal plasma flow and glomerular filtration rate (GFR), and triggers tubular regeneration. Renal repair recapitulates some aspects of embryonic kidney development. Many renal developmental genes are re-expressed and the microenvironment is in favor of cell growth (Supavekin et al., 2003). The inventors and others have shown that intra-renal cells are the main source for regenerating cells (Lin et al., 2003; Lin et al., 2005; Duffield and Bonventre, 2005; Kale et al., 2003; Togel et al., 2005a; Morigi et al., 2004). Recently, Humphreys et al. (2008) genetically labeled 94-95% of the epithelial cells in the nephron proper and further demonstrated that tubular regeneration by surviving intra-tubular cells is the predominate mechanism of adult kidney repair.

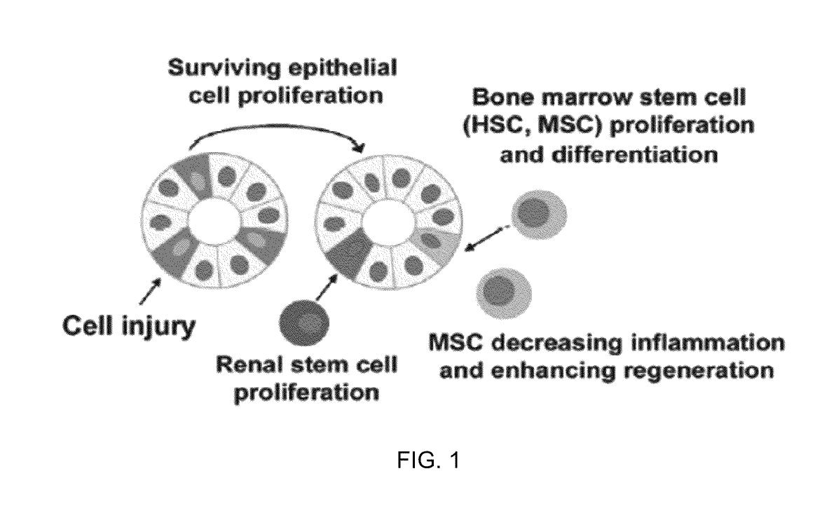

However, bone marrow stem cells which include hematopoietic stem cells (HSC) and mesenchymal stem/stromal cells (MSC) may contribute to renal repair. HSC can be converted to renal cells, whereas MSC can either be converted to epithelial cells or secrete factors to decrease inflammation and enhance regeneration (FIG. 1) (Lin et al., 2005; Duffield et al., 2005; Togel et al., 2005a; Togel et al., 2007). The inventors have previously shown that freshly isolated hematopoietic stem cells (HSC) can incorporate into regenerating kidney tubules. However, the rate of incorporation is low, and no functional benefit is observed.

To enhance the therapeutic potential of hematopoietic stem/progenitor cells, the inventors have developed a novel approach in which hematopoietic stem/progenitor cells are treated to induce renal differentiation prior to transplantation. Importantly, administration of kidney cells generated by in vitro differentiation of hematopoietic stem/progenitor cells has not been utilized for the treatment of any of the above listed renal disorders, MOF or wound healing until the present invention.

In the kidney, the administration of renal cells, derived from hematopoietic stem/progenitor cells by the methods disclosed in the present invention, can be utilized for repair of damaged kidney tissues. The physical or functional loss of reno-vascular endothelial and tubular cells and thus renal function, whether occurring in acute or chronic renal failure, is a serious medical condition that will be ameliorated by the present invention. Any slowing, arrest, or reversal of the decline in renal function provided by the present invention will be enormously beneficial to the affected patients with ARF, TA-ARF, CRF, or any kidney failure-associated systemic dysfunction, MOF or wound healing.

F. Cardiovascular Diseases

The present invention may also have a significant impact on cardiovascular diseases. The kidney regulates fluid homeostasis and blood pressure. Hypertension due to renal insufficiency causes cardiovascular disease. Conversely, cardiovascular disease leads to renal dysfunction. Severe cardiac compromise impairs renal perfusion and can cause renal ischemic injury. The heart and the kidney depend on each other for normal function. Unfortunately, as many as 17.2% patients who undergo cardiac surgeries develop acute kidney injury. Furthermore, cardiovascular complications are the leading cause of death in patients with chronic renal failure. Diseases of the renal and cardiovascular systems adversely affect each other. Therefore, new therapies for kidney disease in this invention will have clear benefit in cardiovascular treatment.

### V. Therapeutics and Transplantation

The present invention provides cells and methods for transplantation into host organisms. Transplantation of stem cells treated and expanded through exposure to nephrogenic factors (e.g., activin-A, retinoic acid, BMP7) into a host may be used, for example, to provide a source of renal cells, to deliver renoprotective factors (renotrophic factors), to express a gene of interest, and to detect and characterize cell expansion and differentiation in vivo (e.g., to provide detectable cells for testing drugs that influence renal cells in vivo). As such, both human and non-human animal hosts find use in the present invention.

In particular embodiments, where cells are to used for therapeutic purposes, the stem cell may be obtained from the subject in need of treatment, and then after expansion, the resulting stem cell expanded through exposure to differentiation growth factors (e.g., nephrogenic factors and epithelial growth factors) and/or HDAC inhibitors in vitro is placed back into the host (e.g., see WO 99/61589 for methods of reintroduction into hosts, herein incorporated by reference in its entirety).

In certain embodiments, a therapeutically effective dose of stem cells is delivered to the patient. An effective dose for treatment will be determined by the body weight of the patient receiving treatment, and may be further modified, for example, based on the severity or phase of the kidney or other organ dysfunction, for example the severity of ARF, the phase of ARF in which therapy is initiated, and the simultaneous presence or absence of MOF. In particular, about 0.01 to about 5×10⁶ cells per kilogram of recipient body weight will be administered in a therapeutic dose, more preferably about 0.02 to about 1×10⁶ cells per kilogram of recipient body weight will be administered in a therapeutic dose. The number of cells used will depend on the weight and condition of the recipient, the number of or frequency of administrations, and other variables known to those of skill in the art. For example, a therapeutic dose may be one or more administrations of the therapy.

### VI. Adjuvant Therapy

In order to increase the effectiveness of renal therapy disclosed in the present invention, it may be desirable to combine these methods with other renal repair/treatment methods, such as renal replacement therapy. Renal replacement therapy is a term used to encompass life-supporting treatments for renal failure. It may include hemodialysis, peritoneal dialysis and hemofiltration.

A. Dialysis

Current major treatment options for patients with severe kidney failure are dialysis and kidney transplantation. Dialysis is a treatment that removes substances such as water, salts, and waste products (from the body's normal metabolism), which build up in patients with failing kidneys.

There are two forms of dialysis. One is called hemodialysis (HD), where the blood is cleaned outside the body and then returned back to the body. This treatment, done in a hospital, or a dialysis clinic, is normally done 3 times a week, where each session takes about 4 hours. In HD, a machine and a filter are required, as well as a system to get the blood out of the body, as well as returning the cleaned blood to the patient. In most patients, this so called access to the blood, is done by inserting two needles into blood vessels on the forearm. A few centers around the world can train patients for self-HD. In regard to transplantation, the new kidney can come from either a deceased person or a living donor.

Hemodialysis (also haemodialysis) is a method for removing waste products such as potassium and urea, as well as free water from the blood when the kidneys are in renal failure. Hemodialysis is one of three renal replacement therapies (the other two being renal transplant; peritoneal dialysis). Hemodialysis can be an outpatient or inpatient therapy. Routine hemodialysis is conducted in a dialysis outpatient facility, either a purpose built room in a hospital or a dedicated, stand alone clinic. Less frequently hemodialysis is done at home. Dialysis treatments in a clinic are initiated and managed by specialized staff made up of nurses and technicians; dialysis treatments at home can be self initiated and managed or done jointly with the assistance of a trained helper who is usually a family member.

The other form of dialysis is called peritoneal dialysis (PD). More than 150,000 patients are currently receiving this lifesaving treatment around the world. In PD, a dialysis fluid is entered into the patient's abdominal (i.e., peritoneal) cavity, which is covered by a thin membrane, containing many small blood vessels. This membrane, called the peritoneum, is like a big bag that keeps the stomach, intestines, liver, and other organs in place. The dialysis fluid will make water, salts, and the waste products move from the blood into the fluid (also called solution). This process is called dialysis, and means that the peritoneum works as a dialysis filter. As the fluid gets saturated after a while, the solution must be exchanged regularly.

The fluids contain either sugar (glucose), amino acids, which are building blocks for proteins, or a compound called icodextrin, to remove the water. The sugar solution is the one most commonly used. The glucose “strength” (1.5%, 2.5% or 4.25%) of the solution determines how much water that is removed from the blood; the higher concentration, the greater the water removal. The amino acid solution is used to improve a patient's nutritional condition (as the amino acids are taken up by the body, i.e., they move from the solution to the blood), and/or to reduce the uptake of glucose from the solution. The benefits of the icodextrin fluid is that it removes more water than the glucose solution for longer exchange intervals, and that it is glucose-free, which is advantageous both for the peritoneum and the body as a whole. There are two types of glucose solutions. The main difference is the pH, and the type of substance added to reduce the acidity of the blood, which is common in patients with kidney disease.

B. Hemofiltration

Hemofiltration, also haemofiltration, is a renal replacement therapy similar to hemodialysis which is used almost exclusively in the intensive care setting. Thus, it is almost always used for acute renal failure. It is a slow continuous therapy in which sessions usually last between 12 hr to 24 hr and are usually performed daily. During hemofiltration, a patients blood is passed through a set of tubing (a filtration circuit) via a machine to a semipermeable membrane (the filter) where waste products and water are removed. Replacement fluid is added and the blood is returned to the patient.

As in dialysis, in hemofiltration one achieves movement of solutes across a semi-permeable membrane. However, solute movement with hemofiltration is governed by convection rather than by diffusion. With hemofiltration, dialysate is not used. Instead, a positive hydrostatic pressure drives water and solutes across the filter membrane from the blood compartment to the filtrate compartment, from which it is drained. Solutes, both small and large, get dragged through the membrane at a similar rate by the flow of water that has been engineered by the hydrostatic pressure. So convection overcomes the reduced removal rate of larger solutes (due to their slow speed of diffusion) seen in hemodialysis.

Hemofiltration is sometimes used in combination with hemodialysis, when it is termed hemodiafiltration. Blood is pumped through the blood compartment of a high flux dialyzer, and a high rate of ultrafiltration is used, so there is a high rate of movement of water and solutes from blood to dialysate that must be replaced by substitution fluid that is infused directly into the blood line. However, dialysis solution is also run through the dialysate compartment of the dialyzer. The combination may be useful because it results in good removal of both large and small molecular weight solutes.

### VII. Examples

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Hematopoietic Stem Cells can be Converted into Kidney Cells after Renal Ischemic Injury

Ischemic injury to the kidney produces acute tubular necrosis and apoptosis followed by tubular regeneration and recovery of renal function. Although mitotic cells are present in the tubules of postischemic kidneys, the origins of the proliferating cells were not clear. To test whether murine HSC can contribute to the regeneration of renal tubular epithelial cells, HSC were isolated from male Rosa26 mice that express beta-galactosidase constitutively and were transplanted into female non-transgenic mice after unilateral renal I/R injury. Four weeks after HSC transplantation, beta-galactosidase-positive cells were detected in renal tubules of the recipients by X-Gal staining. PCR analysis of the male-specific Sry gene and Y chromosome fluorescence in situ hybridization (Y-FISH) confirmed the presence of male-derived cells in the kidneys of female recipients. Antibody co-staining showed that beta-galactosidase was primarily expressed in renal proximal tubules (Lin et al., 2003). This study shows that HSC can differentiate into renal tubular cells after renal ischemic injury.

### Example 2

### Intra-Renal Cell are the Major Source for Renal Repair

Bone marrow cells (BMC) can differentiate across lineages to repair injured organs, including the kidney. However, the relative contribution of intrarenal cells and bone marrow cells to kidney regeneration was not clear. The inventors created ischemic-reperfusion injury (IRI) in transgenic mice that expressed enhanced GFP (EGFP) specifically and permanently in mature renal tubular epithelial cells. Following IRI, EGFP-positive cells incorporated BrdU and expressed vimentin, which provides direct evidence that the cells composing regenerating tubules are derived from renal tubular epithelial cells. In BMC-transplanted mice, 89% of proliferating epithelial cells originated from host cells, and 11% originated from donor BMC. One month after IRI, the kidneys contained 8% donor-derived cells, of which 8.4% were epithelial cells, 10.6% were glomerular cells, and 81% were interstitial cells. No renal functional improvement was observed in mice that were transplanted with exogenous BMC. These results show that intrarenal cells are the main source of renal repair, and a single injection of BMC does not make a significant contribution to renal functional or structural recovery (Lin et al., 2005). The results provide the first direct evidence that tubular epithelial cells can de-differentiate and proliferate to repair injured tubules and indicate that intra-renal cells are the major source for renal repair after AKI.

### Example 3

### Bone Marrow Cell Fusion with Tubular Epithelial Cells is not the Primary Mechanism of Cell Conversion

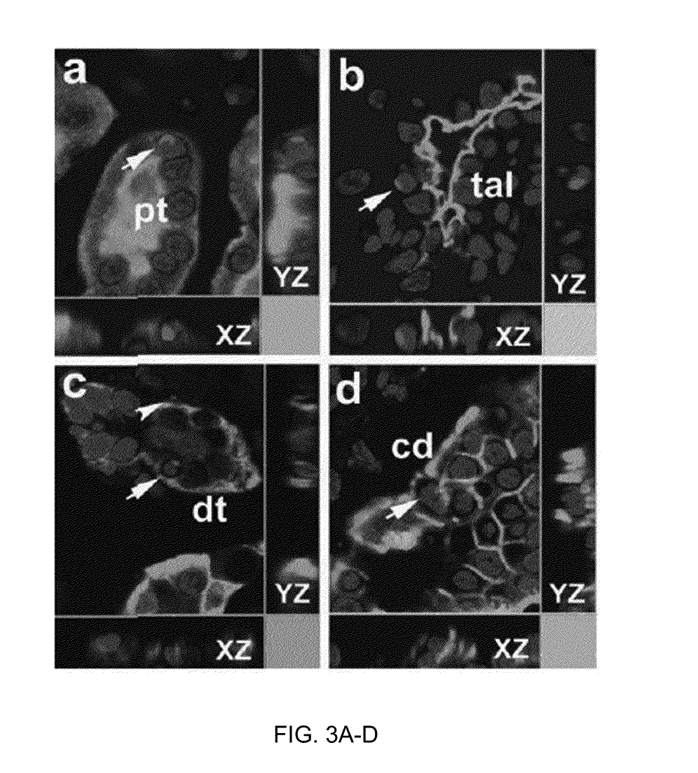

Using laser scanning confocal microscopy, the inventors showed that 1.8% tubular epithelial cells were bone marrow cell-derived and bone marrow cells integrated into all nephron segments of the post-ischemic kidneys one month after injury (Li et al., 2007). The Z-plane images showed the expression of nephron specific markers by bone marrow-derived cells (FIGS. 3A-D).

At one month post IRI, bone marrow cells can also be converted to endothelial cells, mesangial cells and podocytes to a lesser extent. However, most bone marrow-derived cells were localized to the interstitium. Y⁺ cells began to appear in the interstitium and glomeruli as early as 2 days after injury. However, the earliest time when Y⁺ cells could be detected in the tubules was 5 days. At 7 days, only 1 or 2 tubular cells per kidney section were Y⁺. The absence of functional protection may be explained by its minimal conversion to tubular cells in the 1st week post IRI. Further enhancement of early integration and immediate renal conversion may lead to the success of hematopoietic stem cell-based therapy for AKI.

To determine whether cell fusion between bone marrow cells and injured renal cells plays a role, the inventors subjected female mice expressing Cre recombinase in tubular epithelial cells to unilateral renal ischemia-reperfusion injury, and subsequently transplanted male bone marrow cells containing a loxP-flanked reporter gene (R26R-EYFP mice). After 28 days, cell fusion was detected by polysomy for the sex chromosomes and Cre-mediated activation of the reporter gene. After injury, 1.8% of tubular cells were bone marrow-derived, but Cre/loxP recombination demonstrated a frequency of fusion between tubular epithelial and bone marrow cells of only 0.066% (approximately 7 per 10,000 tubular cells). The second strategy, chromosome fluorescence in situ hybridization (FISH), detected cell fusion in 3.8% of the 1.8% bone marrow-derived epithelial cells. No cell fusion was observed in non-ischemic kidneys, suggesting that injury was required for cell fusion. This finding was substantiated in co-culture experiments in which cell fusion was only observed when tubular epithelial cells were depleted of ATP (Li et al., 2007). In conclusion, BMC can fuse with renal epithelial cells after ischemic injury, but the low frequency of fusion does not account for the majority of the observed conversion of bone marrow cells into kidney cells.

### Example 4

### Bone Marrow Cells can be Induced to Adopt a Renal Cell Fate In Vitro

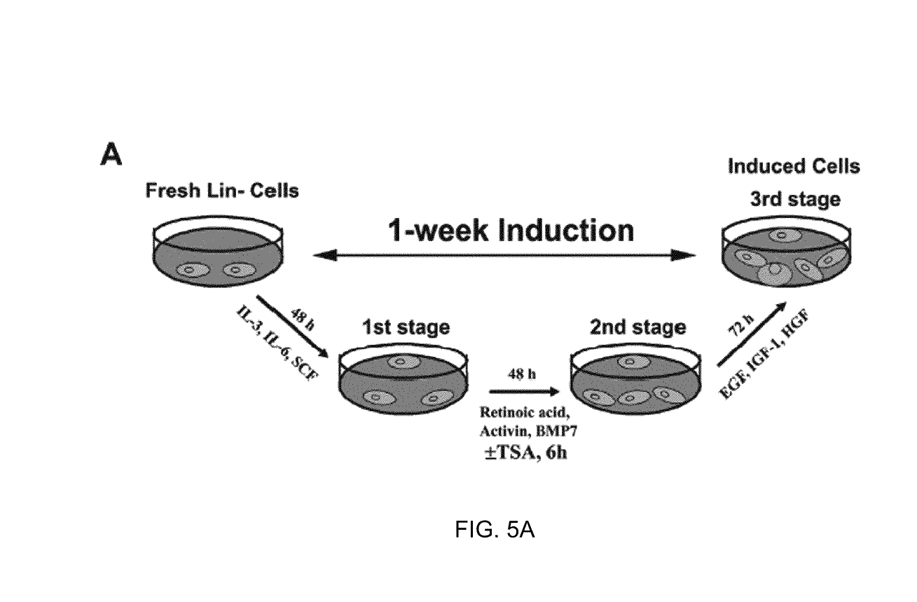

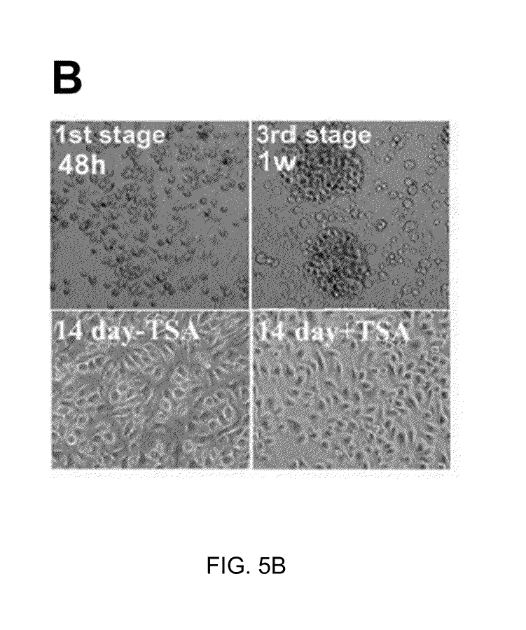

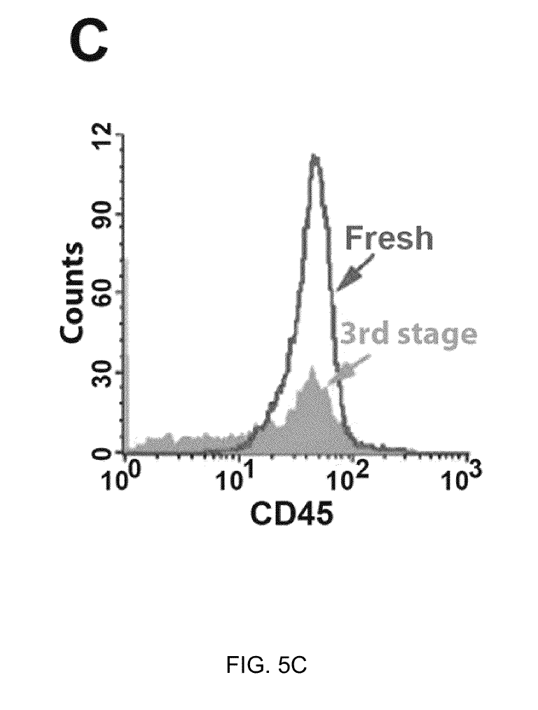

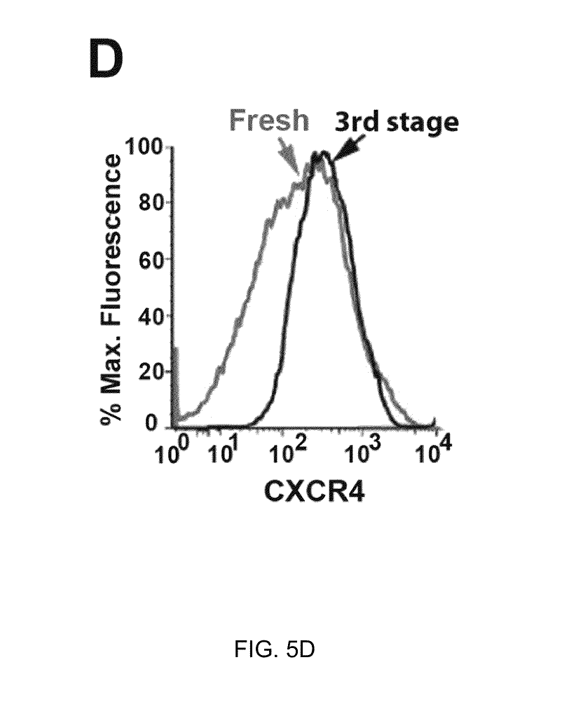

The inventors previously showed that freshly isolated HSC can be transplanted into mice with AKI and are incorporated into regenerating kidney tubules. However, the rate of incorporation is low, and no functional benefit has been observed. To enhance the potential of HSC to contribute to kidney regeneration, the inventors have developed a novel approach in which isolated HSC are treated with growth factors to promote renal differentiation in vitro prior to transplantation. The inventors' approach involves sequential treatment with cytokines, nephrogenic factors, and epithelial growth factors to induce hematopoietic to renal conversion (FIG. 5A).

Lin⁻ cells, which include lineage undifferentiated hematopoietic stem cells and progenitor cells, were isolated to 99.8% purity by FACS as described (Lin et al., 2003). Purified Lin⁻ cells were cultured sequentially in the presence of cytokines including IL-3, IL-6, and stem cell factor for 48 hr (1st stage); nephrogenic factors including retinoic acid, activin-A and BMP7 for 48 hr (2nd stage); and epithelial growth factors including EGF, IGF-1 and HGF for 72 hr (3rd stage). The total induction period is 1 week. Cytokine treatment is known to stimulate HSC to enter the cell cycle and the cells remain as hematopoietic cells after 48 hr in these culture conditions (Luskey et al., 1992; Reddy et al., 1997). The nephrogenic factors that were used can induce differentiation of mouse ES cells into renal epithelial cells as reported by Kim and Dressler (2005). Therefore, the inventors reasoned that nephrogenic factors might induce cycling Lin⁻ cells to select a renal cell fate. Further treatment of the cells with epithelial growth factors would promote epithelial growth. After the 1st stage, the cells remained small, round, and discrete (FIG. 5B, top left). After the 3rd stage, the cells increased in size and formed floating spheres (FIG. 5B, top right). The inventors took the precaution of only transferring cells in suspension to new culture dishes. The adherent cells that might represent bone marrow stromal/stem cells (MSC) were discarded. In some experiments, the inventors continued the cultures for an additional 7 days after the induction period (total 14 days) and observed an epithelial-like morphology in the cells (FIG. 5B, bottom). In the Examples section, the inventors will use the term “induced cells” to refer to cells after the 3rd stage culture (1 week induction).

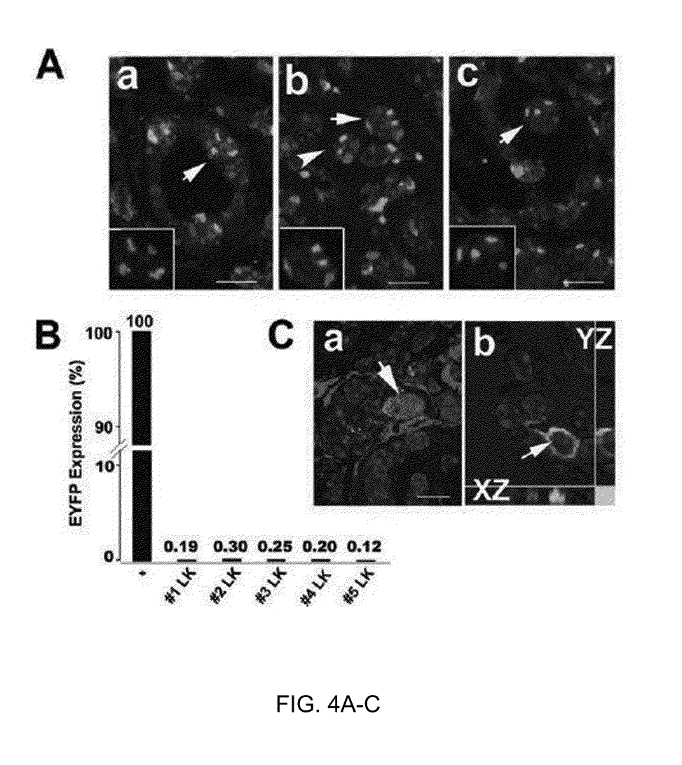

The cells were examined for the presence of hematopoietic specific marker CD45 during the induction period. As expected, freshly isolated Lin⁻ cells expressed CD45. After 1 w induction, most cells retained CD45 on the cell surface (FIG. 4C), indicating incomplete cell fate conversion. Similarly, CXCR4, which is the receptor for stromal derived factor-1 (SDF-1) and plays a critical role in stem cell homing and mobilization, is expressed by 92% of the cells after the 3rd stage induction (n=2) (FIG. 4D). The expression of CXCR4 suggests that the cells might transmigrate into the injured kidney where its ligand SDF-1 is up-regulated.

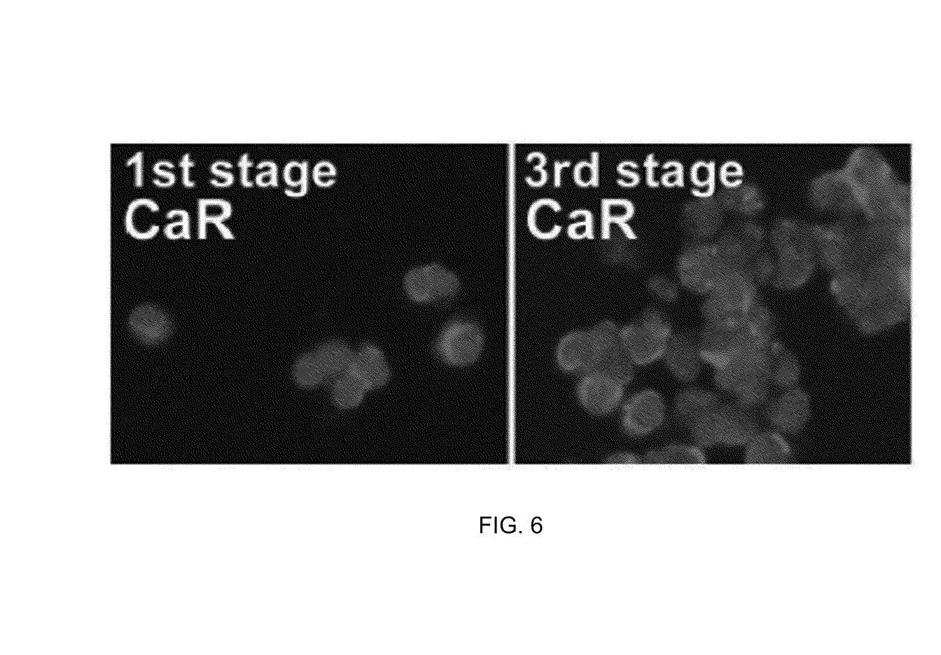

Induced cells continue to express calcium sensing receptor (CaR). Transplanted hematopoietic stem cells (HSC) show preference to home to the bone marrow. One of the homing mechanisms is high Ca²⁺ concentration in the bone marrow endosteum, which attracts HSC to transmigrate to stem cell niche through their surface expression of CaR. Mice deficient in CaR have primitive hematopoietic cells in the circulation and the spleen, but significantly reduced number of HSC in the endosteal niche (Adams et al., 2006). Modulating CaR could alter the distribution of CaR-expressing cells in the bone marrow, systemic circulation and other organs. To examine whether induced cells expressed CaR, immunostaining was performed. The results showed that CaR was expressed on the surface of 97% cells after the 1st stage and 91% cells after the 3rd stage induction (FIG. 6). The inventors contemplate to alter the level of CaR in induced cells to facilitate intra-renal transmigration and decrease bone marrow homing (see Example 8).

Taken together, the morphological changes in the induced cells indicate that Lin⁻ cells may adopt an epithelial-like phenotype under the inventors' induction conditions.

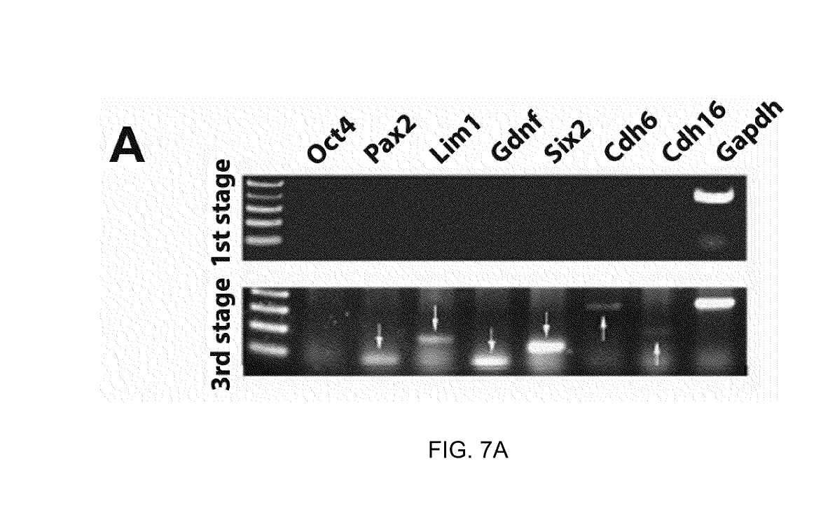

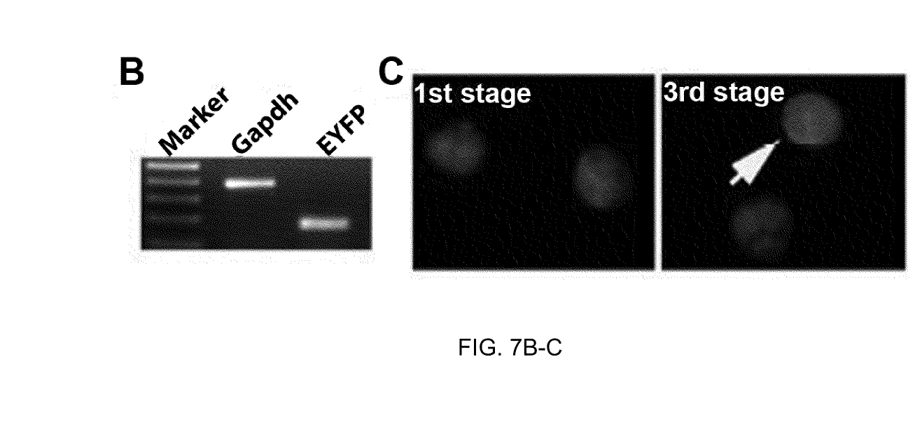

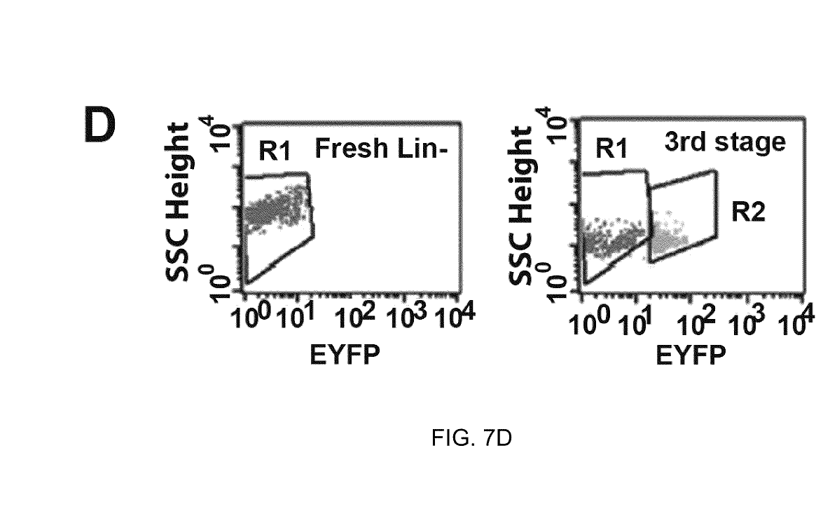

Induced cells select a renal cell fate. The treated Lin⁻ cells were analyzed for the expression of a panel of renal developmental genes by RT-PCR (Kim and Dressler, 2005). The results showed that Oct-4, which is normally expressed in undifferentiated ES cells (Yeom et al., 1996), was not detectable in the cells after the 1st or 3rd stages. In contrast, genes expressed in the developing kidney including Pax2, Lim1, Gdnf, Six2, cadherin-6 and cadherin-16 were expressed after the 3rd stage but were absent after the 1st stage. The above genes were also under detectable levels in cells after the 2nd stage induction. Since cadherin-16 or Ksp-cadherin is selectively expressed in renal epithelial cells, transcription of cadherin-16 in induced cells is highly suggestive of renal fate conversion (FIG. 7A).

To confirm renal conversion, Lin⁻ cells were isolated from bitransgenic creksp; R26R-EYFP mice that express EYFP specifically in the tubular epithelial cells of the kidney. FACS and RT-PCR analyses showed no expression of EYFP in freshly isolated Lin⁻ cells and cells before the 3rd stage induction. However, after the 3rd stage, the induced cells expressed EYFP mRNA by RT-PCR analysis (FIG. 7B) and EYFP protein by immunostaining (FIG. 7C). FACS showed that an average of 6.3% expressing EYFP (FIG. 7D, n=3). Taken together, these results provide further evidence for the activation of a renal program in the induced cells.

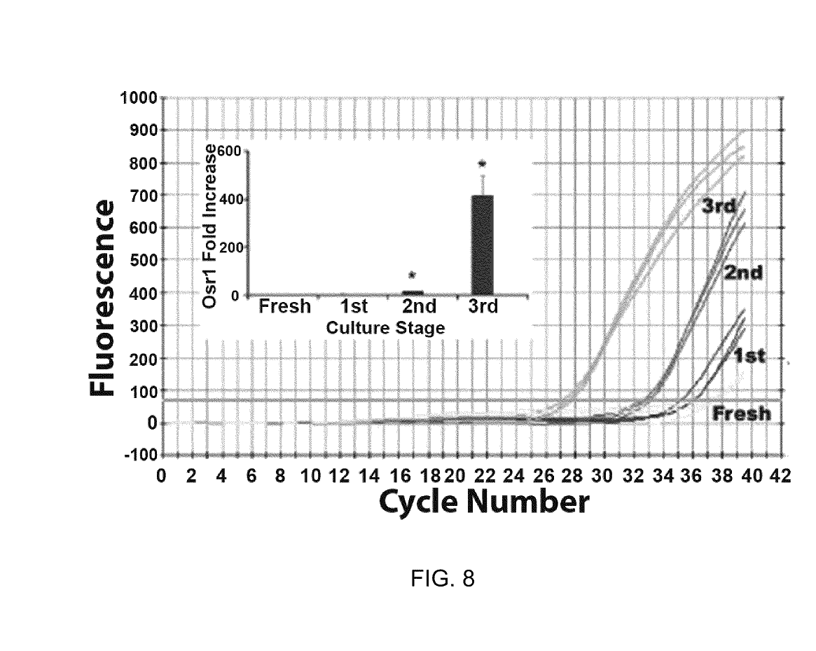

Osr1 is expressed in induced cells. Odd-skipped related 1 (Osr1) is a zinc finger-containing transcriptional factor and is the earliest markers of the intermediate mesoderm that contains precursor cells of all kidney tissue. Osr1 is required for formation of metanephric mesenchyme and activation of a set of renal development genes, such as Pax2 and Lim1 (James et al., 2006). To investigate whether Osr1 gene is expressed during hematopoietic-to-renal conversion, qRT-PCR analysis was performed in freshly isolated Lin⁻ cells as well as cells at each stage of induction. Osr1 was not expressed in fresh Lin⁻ cells. Its level of expression was barely detectable in cells after the 1st stage culture. It started to express at low level after the 2nd stage and its expression increased dramatically after the 3rd stage (FIG. 8). Since other renal genes examined above are only expressed after the 3rd stage, the presence of Osr1 mRNA after the 2nd stage suggests that Osr1 might be the upstream of other renal transcription factors in the inventors' system.

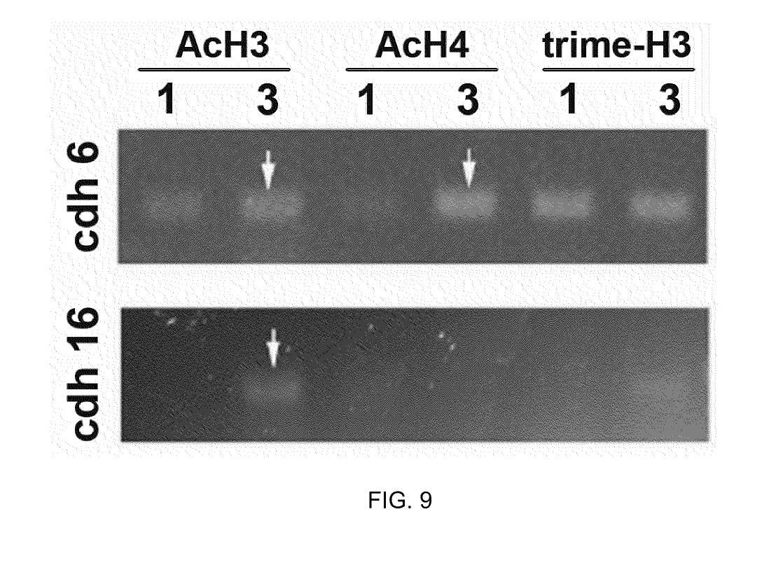

Increased histone acetylation accompanies cell conversion. To begin to understand the mechanisms of cell conversion and its associated nuclear reprogramming, the inventors examined the possibility of epigenetic regulation by histone modifications and chromatin remodeling. Chromatin modifications were detected using chromatin immunoprecipitation assays (ChIP assays) as described (Shang et al., 2000). Antibodies to acetylated histones H3 and H4 and tri-methylated histone H3 (lysine 9 or K9) were used for immunoprecipitation. As shown in FIG. 9, the acetylation of histones H3 and H4 on the cadherin 6 promoter was increased after the 3rd stage. In contrast, there was no change in tri-methylation of H3K9 on the cadherin 6 promoter (top panel). Acetylation of H3 but not H4 on the cadherin 16 promoter was increased after the 3rd stage. Tri-methylation of H3K9 on the cadherin 16 promoter seemed to increase after the 3rd stage. Control experiments using nonspecific IgG showed no changes in histone acetylation, indicating the specificity of the ChIP assay. Taken together, these results indicate that increased histone acetylation and chromatin remodeling accompany the activation of renal gene expression and cell conversion.

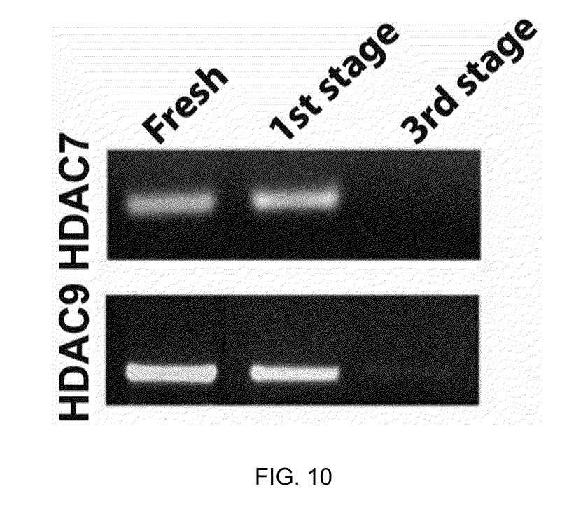

Decreased expression of class II HDACs in induced cells. The level of histone acetylation depends on the opposing activities of HATs and HDACs. Unlike class I HDACs (HDAC1, 2, 3, 8 and 11) that are expressed ubiquitously, class II HDACs (HDAC4, 5, 7, and 9) display tissue-specific expression patterns (Verdin et al., 2003). The expression of class II HDACs in hematopoietic stem cells and progenitor cells has not been carefully studied. The inventors chose to examine the expression of HDAC5, 7 and 9 because HDAC4 null mice die before weaning. Both HDAC7 and HDAC9 were expressed in fresh Lin⁻ cells and cells after the 1st stage of culture. HDAC7 expression was undetectable and HDAC9 expression was dramatically decreased after the 3rd stage induction (FIG. 10). HDAC5 expression could not be detected in fresh or induced cells. These results provide further evidence of increased histone acetylation and its associated activation of renal gene transcription in induced cells.

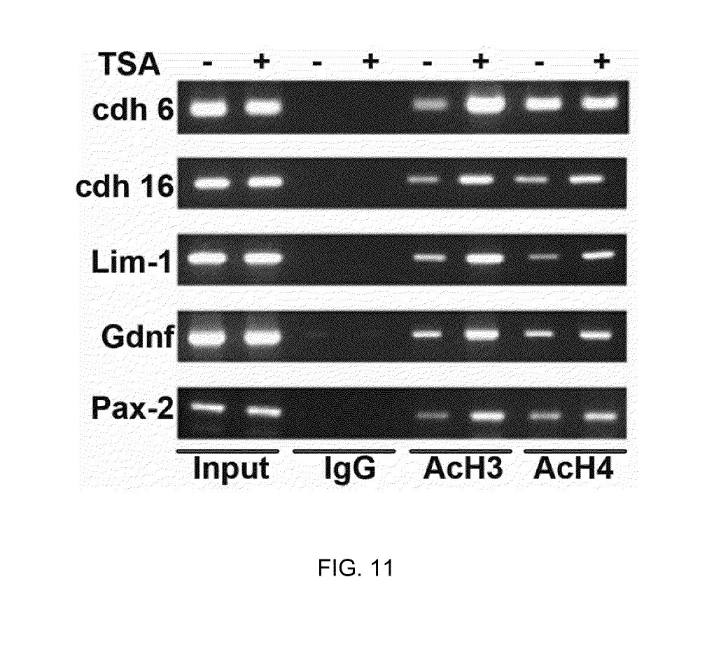

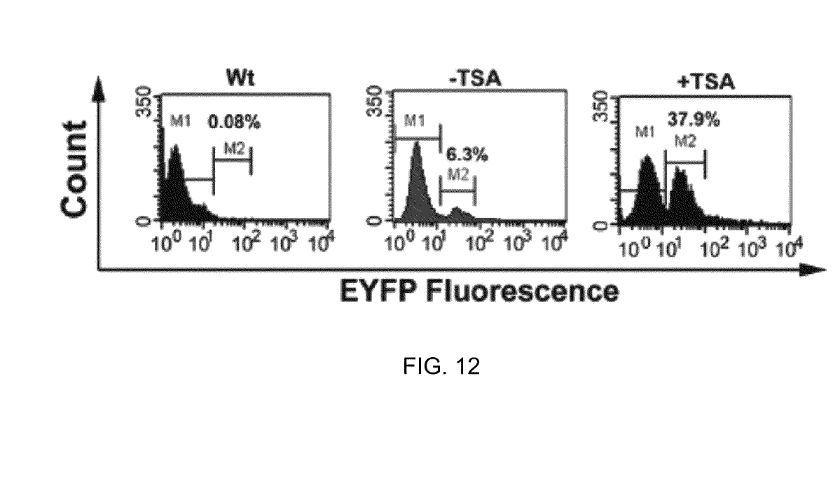

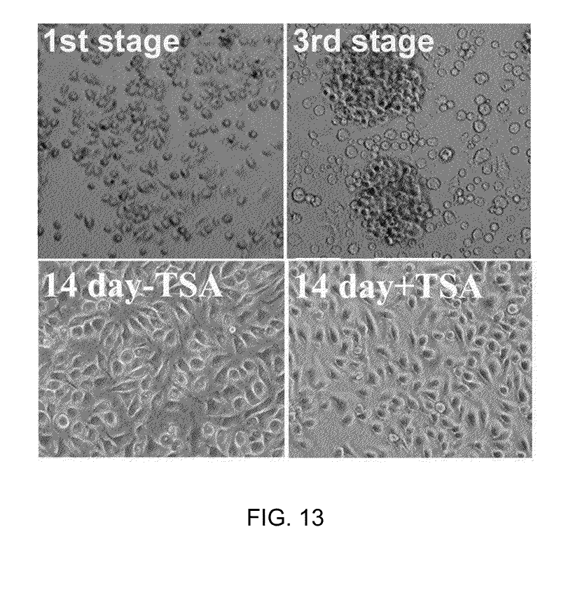

The HDAC inhibitor TSA increases expression of renal developmentally regulated genes in induced cells. Although HDAC inhibitor trichostatin A (TSA) is a non-selective inhibitor of HDACs, TSA only alter 2-10% of the genes tested in multiple cell lines (Gray et al., 2004; Lee et al., 2006b; Mitsiades et al., 2004; Peart et al., 2005). To examine whether inhibition of HDACs increases histone acetylation on cadherin 6, cadherin 16, Lim1, Gdnf and Pax2 promoters, the cells in the 2nd stage were treated with TSA (15 nM, 6 h) or vehicle, and ChIP assay was performed in cells after the 3rd stage induction. The results showed increased histone H3 and H4 acetylation on the promoters of the genes tested (FIG. 11). Moreover, activation of the promoters is associated with the increased expression of renal developmentally regulated genes analyzed by qRT-PCR analysis, while no change of 18s RNA gene was detected (Table 1, *p<0.05, n=3), suggesting selective alternation in gene expression.

TABLE 1
TSA increases renal gene expression in induced cells

| | | | | | | | | 18s |
|---|---|---|---|---|---|---|---|---|
| | Cdh 6 | Cdh 16 | Lim1 | Gdnf | Pax2 | Six2 | Osr 1 | RNA |
| -Fold | 16.5* | 4.39* | 8.29* | 5.39* | 3.28* | 1.3 | 14.95* | −0.06 |
| increase | | | | | | | | |

Treatment with TSA results in a 6-fold increase in EYFP⁺ cells. Lin⁻ cells isolated from cre^{ksp}; R26R-EYFP mice were treated with vehicle or TSA (15 nM, 6 h) at the 2nd stage and EYFP⁺ cells were quantified by flow cytometry analysis. Following TSA treatment, 37.9% of the cells after the 3rd stage were EYFP⁺ as compared to 6.3% in vehicle treated group (6-fold increase), further supporting the importance of chromatin remodeling in cell conversion.

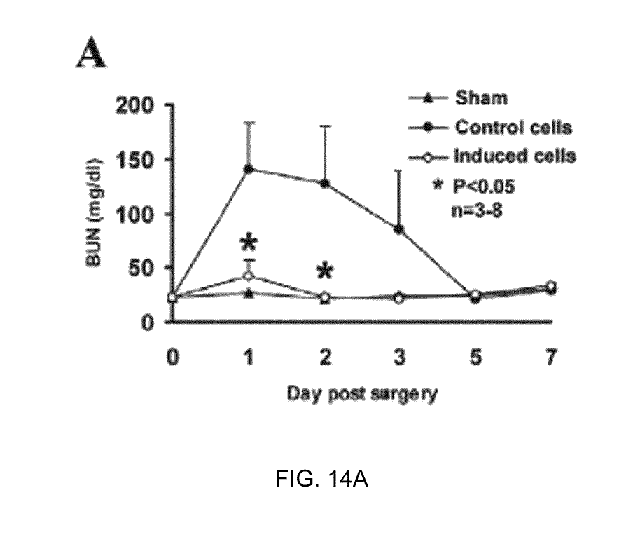

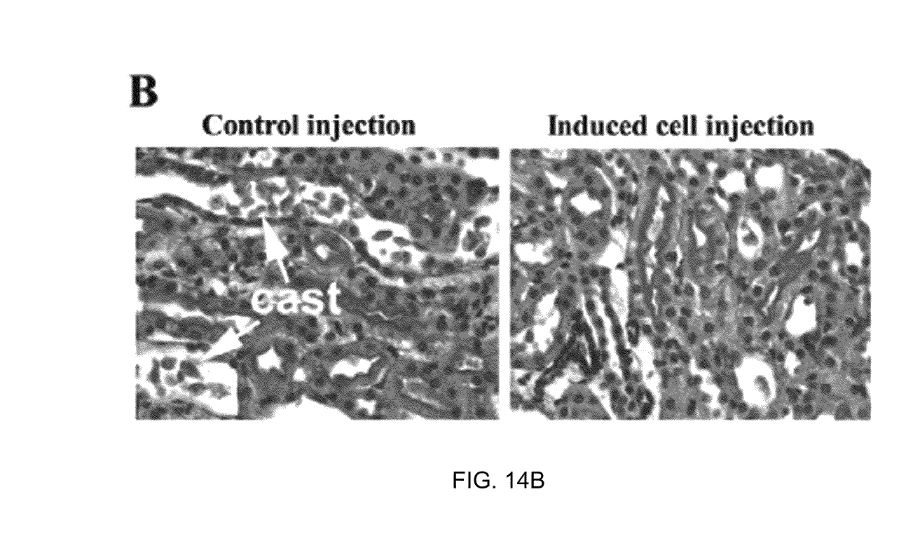

TSA-treated cells provide renal functional and structural protection. To test whether induced cells that were treated with TSA can protect the kidney from AKI, the cells were injected intravenously into mice with right nephrectomy and left renal IRI (or sham-operation as a control). Administration of 5×10⁶ induced cells significantly decreased BUN at 1 and 2 days after injury (FIG. 14A). Renal morphology was also improved (FIG. 14B). Fewer casts and more normal appearing proximal tubules were observed 7 days after injury.

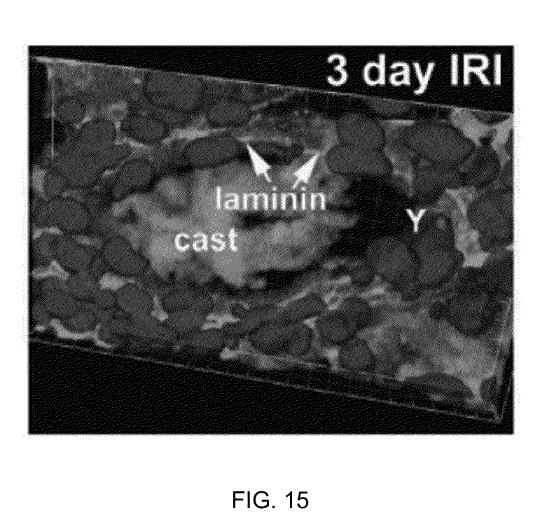

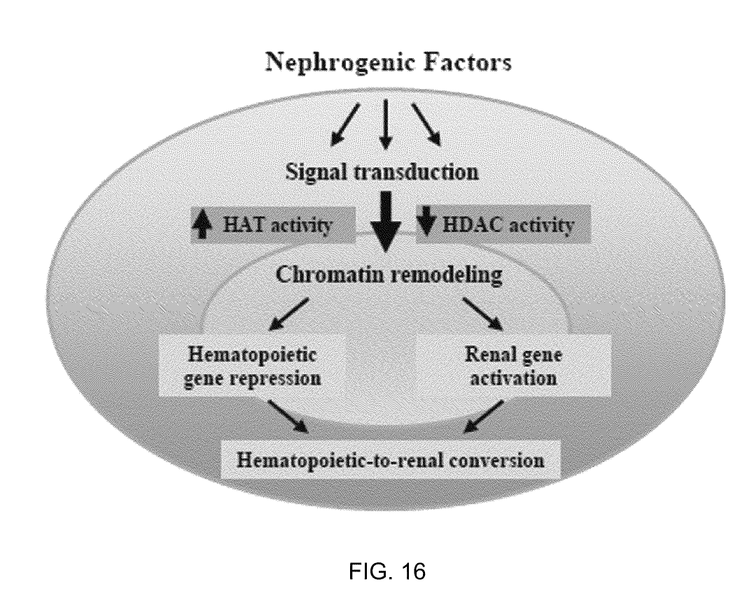

Induced cells integrate into tubules early. Sex-mismatched experiments revealed integration of the transplanted cells into the tubules as early as 3 days post IRI (FIG. 15). Y⁺ cells were also observed in the tubules 7 days post IRI. 0.14% and 0.22% of the tubular cells were Y⁺ at 3 and 7 days post-injury, respectively. No Y⁺ cells were detected in the tubules in sham-operated kidneys. Previously, when freshly isolated and unfractionated bone marrow cells were injected, the earliest time the inventors could observe any integration of male-derived cells into the tubules was 5 days. At 7 days, only 1 or 2 male cells could be detected in all tubules of the entire kidney sections. Early and higher number of tubular integration by induced cells suggests that induced cells can be modified in vitro to increase the efficiency for tubular integration and repair. These results suggest that transplanted induced cells may differentiate into tubular epithelial cells to contribute to structural and functional recovery. However, the possibility that paracrine effects reduce tubular injury and promote intrinsic renal cell proliferation also exists.

### Example 5

### The Molecular Pathways in Hematopoietic-to-Renal Conversion

Lin⁻ cells treated with cytokines, nephrogenic factors and epithelial growth factors differentiate specifically into renal cells in vitro. Induced cells adopt a renal cell fate as evidenced by expression of a panel of renal developmental genes and kidney-specific cadherin-16. The resulting cells in the 3rd stage are heterogenous composed of a mixture of EYFP⁺ and EYFP⁻ cells. Numerous studies have shown that the cytokines used in the 1st stage culture stimulate hematopoietic stem/progenitor cells to enter the cell cycle while maintaining their hematopoietic phenotype (Luskey et al., 1992; Reddy et al., 1997; Wineman et al., 1993). Nephrogenic factors may be the key inducers that initiate the renal program via activation of specific signaling pathways and transcription factors, especially BMP7.

Unlike iPS cells induced from fibroblasts by retroviral delivery of reprogramming factors, Lin⁻ cells and the cells in all stages of the inventors' induction show no expression of Oct4, indicating that hematopoietic-to-renal conversion does not involve a pluripotent ES cell-like state. Reprogramming factors expressed in the fibroblasts are thought to initiate a sequence of stochastic events such as chromatin modification and changes in DNA methylation (Jaenisch and Young, 2008).

Similar stochastic events may occur in the 2nd stage when cycling cells are treated with nephrogenic factors (e.g., retinoic acid, activin-A and BMP7). In response to nephrogenic factors, the cells in the 2nd stage initiate transcription of intermediate mesoderm marker Osr1, which lays upstream of other renal development genes including Pax2, Gdnf and Lim1 expressed in the cells after the 3rd stage induction. The signaling pathways that transmit the stimulation of nephrogenic factors for cell conversion may be revealed by comparing the gene expression (RNA or protein) profile of the cells at each stage of induction, or involve known genes that are specific to hematopoietic stem cells, early kidney development, kidney injury/repair, and pathways related to chromatin remodeling and DNA methylation. The inventors also contemplate up-regulation of transcription factors to initiate renal programming and down-regulation of genes for hematopoietic self-renewal and lineage differentiation during induction.

Treatment with TSA stimulates cell conversion as reflected by increased EYFP expression. TSA causes selective alterations in gene expression (Van Lint et al., 1996; Gray et al., 2004; Lee et al., 2006; Mitsiades et al., 2004; Peart et al., 2005). In numerous cell lines, the expression of only 2-10% genes is altered. While the expression of most renal genes tested is increased, 18s RNA does not change. Although the mechanism is poorly understood, this selectivity has proven to be useful in inducing differentiation of stem cells to specific cell types, such as insulin-producing cells or neuronal cells (Tayaramma et al., 2006; Balasubramaniyan et al., 2006). In the inventors' cultures treated with TSA, increased acetylation of histones on the promoters of several renal genes was associated with a dramatic increase in the number of EYFP⁺ cells. TSA treatment may induce broad activation of gene expression or specific activation of renal genes.

The cells in the final stages of induction will have a renal cell phenotype as preliminary studies show renal gene expression in a mixed cell population by RT-PCR analysis. For example, the induced renal cells may have molecules associated with kidney development including Pax2, GDNF, Lim1, and Six2 which could be detected by immunoblotting or other similar methods. Furthermore, the cells will have a renal epithelial phenotype with cadherin-16 or Ksp cadherin that is known to be expressed specifically in renal epithelial cells. The inventors expect that the expression of Pax2, GDNF, Lim1, and Six2 precedes cadherin-16 that is expressed only after metanephric mesenchyme has undergone epithelial transition. It is expected that EYFP⁺ cells may express cadherin-16 protein because expression of EYFP indicates activation of Ksp promoter. In comparison, some EYFP⁻ cells may be in the early phase of renal conversion and may express Pax2, GDNF, Lim1, or Six2 but not cadherin-16.

In addition, some induced cells retain the expression of CD45, CXCR4 and CaR that are known to be expressed in hematopoietic cells. Loss of expression of CD45 in cells expressing renal proteins will suggest complete hematopoietic-to-renal conversion. The presence of renal proteins and CD45 in the same cell will suggest partial conversion of the cells. Renal epithelial cells are also known to express CaR. Co-expression of CaR and renal proteins may indicate renal conversion or incomplete reprogramming.

The inventors treated Lin⁻ cells with cytokine to stimulate cell cycle entry. This treatment is known to maintain the cells as hematopoietic (Luskey et al., 1992; Reddy et al., 1997; Wineman et al., 1993). Further treatment of the cells with nephrogenic factors (retinoic acid, activin-A and BMP) in the 2nd stage induced low levels of Osr1 expression (FIG. 8), which is believed to be the upstream of other renal developmental genes such as Pax2 and Lim1 (James et al., 2006). Nephrogenic factors have been shown to induce mouse ES cells to express markers specific for intermediate mesenchyme. The treated cells differentiated into renal epithelial cell efficiently (Kim and Dressler, 2005). It is likely that nephrogenic factors are the key inducers in hematopoietic-to-renal conversion.

Retinoic acid regulates patterning and development of many organ systems including the kidney (Mendelsohn et al., 1999; Osafune et al., 2002). It binds to nuclear receptors and regulates gene expression via interaction with coactivators and corepressors. Most of the coregulators are involved in modification of chromatin and nucleosome structure (McGrane, 2007). On the other hand, activin-A and BMP7 belong to TGF-β superfamily and exert their biological activity through TGF-β signaling pathway. TGF-β signaling pathway plays an important role in kidney development and renal repair after injury (Cain et al., 2008; Vukicevic et al., 1998). Members of the TGF-β superfamily can exhibit disparate effects on cell fate commitment and differentiation (Kitisin et al., 2007). It is likely that the combination of growth factors and cross-talk among the intracellular signaling pathways determine the nuclear transcription program and hematopoietic-to-renal conversion.

The inventors may also determine other signaling pathways which may promote renal conversion by methods known in the art. For example, the inventors can use the system of pathway-focused gene expression profiling that is available from SuperArray Bioscience (Frederick, Md.). This system combines the quantitative renal-time PCR with the multiple gene profiling capacity of a microarray. Each PCR array contains 84 or 370 genes related to a specific pathway. The set of primers can also be customized for specific genes known to be involved in kidney development and repair. This system will complement Illumina global gene array due to its sensitivity, accuracy and quantitative nature. It is expected that this system will be able to detect initial small changes in gene expression in the 2nd stage culture.

Cells in the 1st, 2nd and 3rd stage cultures will be harvested and gene expression will be analyzed using PCR array sets of a) transcription factors; b) signal transduction pathway finder; c) TGF-β BMP signaling pathway; and d) customized renal developmental genes. The customized set of genes will be selected from the results of Illumina global gene array study and the published results of Genitourinary Development Molecular Anatomy Project (GUDMAP, available through world wide web at gudmap.org) (Little et al., 2007). The level of gene expression at each stage induction will be compared and an integrative pathway connection map will be generated.

The global gene expression profiling and defining of specific signaling pathways and their associated activation of transcription factors may provide additional renal differentiation factors that could promote renal conversion. Transplantation of induced cells may enhance renal repair from endogenous cellular source in a paracrine fashion by providing renal protective factors that could stimulate intrinsic tubular epithelial proliferation.

### Example 6

### The Mechanisms of Cell Conversion

Conversion of Lin⁻ cells to renal cells depends on histone acetylation and chromatin remodeling. Inhibition of histone deacetylation with siRNA or gene targeting of HDAC7 and HDAC9 will enhance the conversion of Lin⁻ cells to renal cells.

Detailed profiling of gene expression indicates that lineage undifferentiated hematopoietic cells support transcription of non-hematopoietic genes (Akashi et al., 2003). A combination of IL-3, IL-6, and SCF causes HSC to enter the cell cycle and complete the first division within 36-40 hours (Reddy et al., 1997). The changes of chromatin structure associated with cell cycle progression opens the window for transcriptional regulation (Akashi et al., 2003; Cerny and Quesenberry, 2004; Reddy et al., 1997; Passegue et al., 2005; Becker et al., 1999; Dooner et al., 2004; Lambert et al., 2003; Quesenberry, 2006). After treatment with cytokines, exposure of Lin⁻ cells to nephrogenic factors followed by epithelial growth factors activates the expression of a set of genes important in kidney development.

Histone acetylation mediated by HATs relaxes the compact chromatin structure making DNA more susceptible to transcriptional activation. In contrast, histone deacetylation is associated with gene repression. The level of histone acetylation depends on the opposing activity of HATs and HDACs. The inventors' preliminary results show that the expression of renal genes coincides with the increased acetylation of histone H3 on the promoters of a set of renal developmental genes. Inhibition of HDACs with TSA further increases histone acetylation on the promoters.

The levels of histone acetylation, primarily as the result of changes in HAT and HDAC activity, may cause chromatin structure changes which then initiate transcription of renal genes while repressing hematopoietic genes. The relatively low induction (6.3% EYFP⁺ cells) in the absence of a HDAC inhibitor TSA may be explained by the fact that nuclear reprogramming in response to environmental factors is stochastic. Forcing histone acetylation with TSA amplifies the chain of nuclear events, which results in dramatic increase in cell conversion (37.9% EYFP⁺ cells). More converted cells will therefore be available for transplantation studies to test their ability in treating mice with AKI.

The increase of histone acetylation occurs in the setting of decreased expression of HDAC7 and 9. These results suggest that increased histone acetylation may activate renal gene expression and promote cell conversion. Therefore, modulation of tissue specific HDACs, such as HDAC7 and 9 may help hematopoietic-to-renal conversion.

Class II HDACs, such as HDAC9, contain an N-terminal domain that interacts with other transcriptional coactivators to confer responsiveness to extracellular signals (Verdin et al., 2003). The N-terminal domain also contains two conserved phosphorylation sites for calcium/calmodulin-dependent protein kinase (CaMK). Phosphorylation of these sites promotes binding to 14-3-3 proteins, which induce nuclear export and derepression of target genes (McKinsey et al., 2000). Conversely, mutation of the phosphorylation sites causes retention of HDACs in the nucleus and significantly reduces histone acetylation and represses target genes (Zhang et al., 2002).

Particularly, negative modulators of HDAC7 and HDAC9 may be used because of the dramatic reduction in their expression in cells after the 3rd stage induction. The inventors contemplate promoting renal cell conversion by introducing a dominant negative HDAC7 or HDAC9 mutant, a HDAC7/HDAC 9 siRNA or antisense RNA into cells by a vector to inhibit HDAC9 function. The inhibition of HDAC7 and/or HDAC9 will lead to increased levels of renal gene expression and histone acetylation, and increased percentages of induced renal cells.

Alternatively, CBP and P/CAF that have intrinsic HAT activity may be over-expressed in the cells or their agonists may be used to treat the cells to increase histone acetylation and renal cell conversion.

In addition, the inventors may also use a combination of histone acetylation modulators described above to further enhance renal cell induction synergistically.

### Example 7

### Renal Protection by Induced Cells

Induced cells that have been transplanted can integrate into renal tubules and differentiate into functional epithelial cells that express markers of well-differentiated cells, such as membrane transport proteins. Induced cells may also provide renal protection by decreasing renal injury and promoting intrinsic renal cell proliferation in a paracrine fashion. Transplantation of induced cells is safe and effective.

The mechanism of renal protection afforded by the induced cells may include (1) direct differentiation of the cells into tubular epithelial cells; (2) paracrine effects that decrease renal injury and promote intrinsic renal cell proliferation, as suggested by mesenchymal stem/stromal cells (Togel et al., 2005a; Togel et al., 2007; Bi et al., 2007); and (3) both cell differentiation and paracrine effects. The detection of transplanted cells in the tubules 3 and 7 days after injury suggest that the cells may differentiate promptly into tubular epithelial cells. The improvement in renal function one week after ischemia-reperfusion suggests less cell injury or improved regeneration by surviving epithelial cells. The fact that induced cells express a panel of transcription factors that are involved in kidney development suggests that combination of the transcription factors may enhance proliferation and differentiation of intrinsic renal cells that have been shown to be the major source for regenerating tubular cells.

Without TSA treatment, 6.3% of HSC differentiate into cells that express EYFP, indicating activation of the kidney-specific cadherin-16 promoter. With TSA treatment, the number of EYFP⁺ cells is increased 6-fold. The protective effects of TSA-treated cells could be due to the higher number of EYFP⁺ cells in the culture or could reflect effects of TSA that are independent of cell number.

The inventors' preliminary transplantation studies were performed using the entire population of induced cells, which includes both EYFP⁺ cells and EYFP⁻ cells. It is possible that some EYFP⁻ cells are in the continuum of adopting a renal cell fate and might develop into renal cells once located in an appropriate environment.

Use of HDAC inhibitors may take into account the fact that high dose and/or long period of HDAC inhibition may induce widespread changes in gene expression. For example, treatment of metanephric organs cultures with HDAC inhibitor induces significant disturbance in the transcription network (Chen et al., 2007). However, inhibition of HDAC activity by TSA (IC₅₀ in nanomolar range) is reversible in cell cultures and in vivo (Vigushin et al., 2001). The inventors only treated the cells with a low concentration (about 15 nM) for a relatively short period of time (6 hr) in the 2nd stage and no increase in cell death was observed. Instead, more hematopoietic cells converted to renal cells in the 3rd stage, suggesting no short-term toxicity.

The following example provides a method that induced renal cells may be used for renal protection in mice. Lin-cells will be purified from whole bone marrow cells by FACS as described (Lin et al., 2003). Purified Lin⁻ cells will be cultured sequentially in the presence of cytokines for 48 hr (1st stage); nephrogenic factors for 48 hr (2nd stage); and epithelial growth factors for 72 hr (3rd stage) as described in the preliminary studies. In some experiments, the cells will also be treated with 15 nM TSA for 6 hr during the 2nd stage. To monitor the conversion into renal cells, the Lin⁻ cells will be isolated from bitransgenic cre^{ksp}; R26R-EYFP mice that express EYFP (green) specifically in renal tubular epithelial cells. After the 3rd stage induction, expression of EYFP will be measured by qRT-PCR and fluorescence microscopy. EYFP⁺ and EYFP⁻ cells will be sorted by FACS.

EYFP⁻ and/or EYFP⁺ cells with and without TSA treatment will be injected intravenously into mice with renal ischemia-reperfusion injury (IRI). To identify the transplanted cells, cells isolated from male cre^{ksp}; R26R-EYFP mice will be transplanted into female C56BL/6 recipients. This approach will enable the transplanted cells to be identified by Y chromosome FISH. The female mice will be divided into 3 groups: SHAM, IRI with treated HSC (IRI⁺ CELL), and IRI without treated HSC (IRI⁻ CELL). The inventors' preliminary studies showed renal protection following injection of 5×10⁶ TSA-treated cells which contained 37.9% EYFP cells. The dose response will be determined by injecting 2×10⁵, 1×10⁶, and 5×10⁶ EYFP or EYFP⁻ cells 2 hr after renal IRI.

Mice will be given BrdU for 14 days to label proliferating cells. Y-FISH analysis or immunostaining of EYFP combined with nephron segment-specific transporters (Table 2) will be performed to identify donor-derived cells (Lin et al., 2005). Differentiation of induced cells into tubular cells will be identified by co-localization of donor cell markers and tubular transporters. Immunostaining of activated caspase 3 will be performed to detect apoptotic cells. Proliferation of donor cells (BrdU⁺Y⁺) and recipient cells (BrdU⁺Y⁻) will be quantified and compared at 7, 14 and 28 days after IRI. Renal morphology will be evaluated by PAS staining at 3, 7, 14 and 28 days. Renal function will be determined by measurements of serum BUN and creatinine at 1, 2, 3, 5, 7 and 14 days. Some mice will be maintained for 6 months to monitor long-term renal function and morphology.

TABLE 2
Nephron-specific epithelial transporters

| Nephron segments | Antibody to transporters and source |
|---|---|
| Proximal tubules | NaPi-2 (Dr. Heini Murer, University of Zurich) |
| Thick ascending limbs | NKCC2 (Alpha Diagnostic International) |
| Distal tubules and | NCX1 (Dr. Kenneth Philipson, UCLA) |
| connecting tubules | |
| Collecting ducts | AQP3 (Chemicon International) |

If no significant renal protection is observed with certain doses of cells injected at 2 h post IRI, a second intravenous injection will be given at 24 h when renal vasoconstriction is less pronounced (Bonventre, 1993; Yagil et al., 1989). Alternatively, cells may be directly delivered by injection under the renal capsule.

The inventors expect that injected cells will be differentiated as detected by male Y chromosome and expression of epithelial transporters. Paracrine effects from injected cells may play a role for functional protection in combination with renal cell differentiation. TSA treatment may alter cell properties to promote renal function and protection in short-term (within the first 28 days) and long-term (6 months).

Because the induced cells may have endocrine and paracrine effect on kidney repair, the inventors contemplate administration (e.g., peritoneal or intravenous injection) of conditioned medium obtained from the culture of the induced cells. The methods of preparing conditioned medium from cultured cells are known in the art, for example, as disclosed in Bi et al. (2007). The factors produced by the cells may also have beneficial effects in the renal cells injured by toxins, ATP depletion and hypoxia.

Unlike the methods used for generation of iPS cells, the inventors' induction method does not involve retroviral gene delivery and no pluripotent cells are produced as indicated by no expression of Oct4. The inventors do not expect teratoma formation from injected cells. The inventors have excluded MSC in the preparation.

### Example 8

### Enhance the Therapeutic Potential of Induced Cells for Renal Repair

Blocking the calcium-sensing receptor (CaR) on induced cells can promote their intra-renal transmigration to increase tubular integration and/or production of renal protective paracrine factors. This strategy will enhance the therapeutic potential of induced cells to treat AKI.

Most renal regeneration occurs in the first week after IRI (Lin et al., 2005). Early and sustained transmigration of induced cells into the kidney could offer additional cellular source for renal repair. Although small number of hematopoietic stem and progenitor cells can be found in many tissues including the kidney during the first few hours of cell infusion (Papayannopoulou and Craddock, 1997; Kretchmar and Conover, 1969; Vos et al., 1972), HSC engraft preferentially to the bone marrow where stem cell niches are located (Adams et al., 2006; Nilsson et al., 2001).

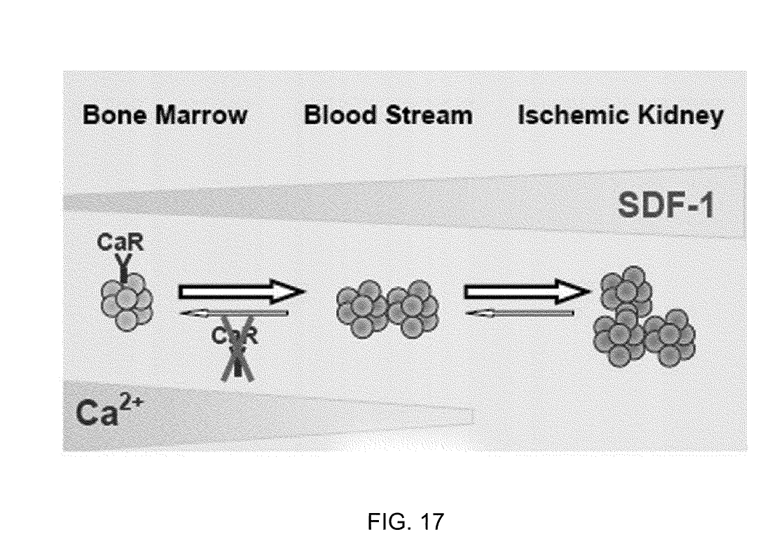

SDF-1 expressed by bone marrow stromal cells and endothelial cells, and CXCR4 expressed on stem and progenitor cells play an important role in homing and mobilization. In the normal kidney, SDF-1 is expressed in the distal tubules. Low level of proximal tubular expression has also been reported. Two to twenty-four hours following ischemic injury, SDF-1 expression is up-regulated and can be detected in most surviving tubular cells. Conversely, SDF-1 protein level decreases in the bone marrow after renal IRI (Togel et al., 2005b). The changes of SDF-1 gradient favor the migration of stem and progenitor cells into the circulation and enter the kidney (FIG. 17).

The inventors' preliminary studies indicate that 92% of induced cells in the 3rd stage retain the expression of CXCR4. In a recent publication, Adams et al. showed that stem cell homing to the bone marrow was mediated by the interaction of CaR expressed on the stem cell surface with the high local Ca²⁺ concentration in the osteoblasts of the bone marrow (as high as 40 mM, more than 20 times of Ca²⁺ concentration in the circulation) (Adams et al., 2006). Hematopoietic stem cells isolated from CaR−/− mice were unable to migrate to endosteal niche of the bone marrow. In contrast, activation of CaR by a selective CaR agonist (calcimemetic) NPS R-467 increases monocyte migration in response to calcium (Olszak et al., 2000). Combined with the increased expression of SDF-1 in the post-ischemic kidneys and decreased expression of SDF-1 in the bone marrow, the inventors contemplated that blocking CaR on induced cells would further reduce homing of the cells to the bone marrow once injected intravenously. As a result, intra-renal transmigration of the cells would increase (FIG. 17). Once in the kidney, induced cells that already selected renal cell fate would integrate into renal tubules more efficiently during the early phase of renal repair. More intra-renally localized cells could also produce higher levels of paracrine factors for better renal protection.

The important role of CaR in mediating monocyte transmigration and HSC homing to the bone marrow has been established (Olszak et al., 2000; Adams et al., 2006). In certain aspects, the inventors contemplate incubating the Lin-cells or the induced renal cells with a CaR blocker, such as a CaR antibody.

Induced cells (10⁴ cells) will be incubated with vehicle or of a mouse monoclonal antibody to CaR (Novus Biologicals) at 1:50, 1:100, 1:200 or 1:400 dilutions for 1 hour prior to transmigration assays. Next, in vivo experiments using CaR antibody on decreasing bone marrow homing and increasing renal transmigration will be performed in mice with renal IRI. Induced cells isolated from male cre^{ksp}; R26R-EYFP mice will be incubated with vehicle or the optimal concentration of the antibody for 1 hr. The recipient female C57BL/6 mice will undergo unilateral renal IRI as described (Lin et al., 2005). The cells will be injected via tail vein into the recipients (5×10⁶ cells/mouse) 2 hours or 24 hours post renal IRI (Togel et al., 2005b). Y chromosome FISH analysis will be performed to quantify the injected cells in the peripheral circulation, bone marrow, and the kidneys at 1, 3, 5, 24, 48 and 72 hr post-transplantation. Cell kinetic studies in stem cell transplanted recipients have shown that the cells appear in the extramedullary tissues (kidney, liver, lung) in the first few hours after injection, but most of the cells home to the bone marrow within hours of intravenous injection (Papayannopoulou and Craddock, 1997; Vos et al., 1972; Nilsson et al., 2001; Laird and von Andrian, 2008). The inventors may extend the time course to 72 hr to examine whether blocking CaR causes prolonged retention of the cells in the circulation and increased transmigration into post-ischemic kidneys.

As an alternative, a potent small molecule CaR antagonist (calcilytic) NPS-2143 (Nemeth et al., 2001) that is near completion in its phase II clinical trial for treatment of osteoporosis (conducted by NPS Pharmaceuticals and GlaxoSmithKline) will be used to block CaR on induced cells prior to cell transplantation.

### Example 9

### Induce Human Umbilical Cord Blood-Derived Hematopoietic Stem Cells (hUCB-HSC) to Differentiate into Renal Cells

One of the best examples of cell conversion is the induction of mouse fibroblasts into ES-like cells by defined transcription factors (Takahashi and Yamanaka, 2006). The same condition also induces human fibroblasts into ES-like cells (Takahashi et al., 2007), indicating conserved mechanism in nuclear reprogramming between mice and humans. The inventors have shown that mouse bone marrow HSC can be induced to become functional renal cells in vitro. The inventors will treat human umbilical cord blood-derived hematopoietic stem cells (hUCB-HSC) with inducers for hematopoietic-to-renal conversion.

Hematopoietic stem cells and progenitor cells in the human umbilical cord blood are CD34⁺ and can be isolated by immunobead methods (Wilpshaar et al., 2000). CD34⁺ cells will be treated sequentially with cytokines for 48 hr (1st stage), nephrogenic factors for 48 hr (2nd stage), and then epithelial growth factors for 72 hr (3rd stage). Some cells will be treated with HDAC inhibitor TSA (15 nM) to increase conversion efficiency for 6 hr at the 2nd stage. Cytokines treatment is known to stimulate cell cycle progression (Luskey et al., 1992; Reddy et al., 1997). The inventors reason that chromatin remodeling during cell cycle progression will open the window of opportunity for cells to respond to nephrogenic factors and differentiate into renal cells. Further treatment with epithelial growth factors will promote renal epithelial growth.

To identify the converted renal epithelial cells, the induced cells will be analyzed by phenotype analysis. Multicolor flow cytometry analysis will be performed to identify cells expressing kidney epithelial specific cadherin (Ksp cadherin) and molecules associated with kidney development, including Pax2, GDNF, Lim1, Six2, or cadherin-6. HSC have been shown to differentiate into cardiomyocytes, neural cells, hepatocytes, pancreatic cells, and endothelial cells. Therefore, cells will be stained with antibodies to myosin heavy chain, nestin, albumin, insulin, and CD31 to identify the possible conversion into these cells. The presence of epithelial markers and renal developmental molecules and the absence of markers of other cell types will indicate selective induction of renal cell conversion.

It is likely that genes involved in blood cell lineage differentiation and HSC self-renewal will be down-regulated, whereas genes expressed in renal epithelial differentiation will be up-regulated. It is also likely that TGF-β signaling pathways will be activated since two of the key nephrogenic factors, activin A and BMP7, are signaled through TGF-β pathways.

Chromatin remodeling is critical for epigenetic control of cell type- and stage-specific gene expression. It is involved in cell conversion and nuclear reprogramming. Histone acetylation associated with chromatin modification usually leads to gene activation. The level of histone acetylation is determined by the activities of histone acetyltransferase and its opposing histone deacetylase (HDAC). In the inventors' studies to induce mouse bone marrow HSC to differentiate into renal cells, an increase in histone acetylation on the promoter of kidney specific Ksp cadherin was detected. Renal gene expression coincides with the decreased expression of HDAC7 and 9. Further treatment of the cells with a non-selective HDAC inhibitor TSA increased the expression of a panel of renal genes by 4- to 15-fold, and cell conversion by 6-fold. These results indicate that hematopoietic-to-renal conversion is accompanied by elevated levels of histone acetylation, hence the critical role of histone acetylation in nuclear reprogramming.

To further enhance renal cell conversion, the inventors will inhibit HDAC7 or 9 with siRNA to specific knocking down of HDAC7 or 9 in hUCB-HSC. The cells will be cultured in the presence of cytokines for 48 hr (1st stage) and transfected with control siRNA or siRNA specific for HDAC 7 or 9 (Chang et al., 2006). Down-regulation of HDAC7 or 9 transcripts and protein will be confirmed by qRT-PCR and immunoblotting. Cells will then be cultured through the 3rd stage. qRT-PCR analysis for a panel of renal gene expression (Pax2, Lim1, GDNF, Six2, and cadherin-6 and 16), chromatin immunoprecipitation (ChIP) assays for histone acetylation, and FACS analysis for isolation of Ksp⁺ cells will be performed. Inhibition of both HDAC7 and 9 using siRNA may also be performed. The induced cells will be transplanted into SCID mice with renal ischemic injury for renal structural and functional recovery. The inventors expect that transient inactivation of HDAC7 and 9 is likely to cause minimal disturbance in cell growth and will have little long-term safety issues.

To treat kidney disease, sorted Ksp⁺ cells that have been treated with vehicle or TSA will be transplanted by intravenous injection into SCID mice with renal ischemia-reperfusion injury (IRI). Alternatively, cells may be injected under the renal capsule for direct renal delivery. The inventors expect that treated hUCB-HSC will contribute to renal protection by direct differentiation of the induced cells into epithelial cells to restore tubular structure and/or paracrine effects that decrease renal injury and promote intrinsic renal cell proliferation.

### Example 10

### Induced Cells Protect Kidney by Producing Renotrophic Factors

Injection of conditioned medium from induced cells accelerates renal recovery. To test whether induced cells can produce endocrine and/or paracrine factors that protect kidney cells from injury and enhance renal recovery, conditioned medium from cell culture was collected. The conditioned medium was injected into the peritoneum of the mice with renal ischemic injury (1 ml, twice a day for 3 days). The inventors observed decreased serum blood urea nitrogen and creatinine in mice injected with conditioned medium. These results support endocrine and/or paracrine effects of induced cells on renal protection.

Induced cells express renotrophic factors. To begin to identify the renotrophic factors produced by induced cells, cDNA microarray analysis was performed. Illumina Mouse-6 Beadchips that contain 47,000 probers/chip were used for hybridization. RNA was isolated from TSA-treated induced cells and fresh Lin⁻ cells. Data were analyzed using more stringent low intensity filtering (detect P value < or =0.01 and fold change > or =2.0). Among differentially expressed genes, a panel of growth factors, growth factor receptors, and binding proteins was upregulated in induced cells (Table 3). These include Igf-1, Hgf and Vegfa that have been shown to have a renotrophic effect. Epithelial markers, such as E-cadherin, laminin and many solute carriers, were also upregulated. In contrast, hematopoietic differentiation genes were significantly down-regulated. These results support hematopoietic-to-renal lineage reprogramming. Interestingly, Klf4, an oncogene used in the induction of pluripotent iPS cells, and chromatin modifying enzymes such as DNA methyltransferase B3 and histone deacetylase 7 (HDAC7) are down-regulated as well, further supporting the importance of histone modification in hematopoietic-to-renal conversion.

Future studies using individual or combination of renotrophic factors may provide additional options for treatment of kidney disease.

TABLE 3
Differential gene expression in induced cells vs. Lin⁻ cells

| Growth factors/ | |
|---|---|
| receptors/ | Fold- |
| binding proteins | increase |
| Igf-1 | 49.44 |
| Igf1bp2 | 5.19 |
| Tgfb1i1 | 4.70 |
| Igf2r | 4.60 |
| Activin-A | 4.33 |
| Egr1 | 4.26 |
| Igf2r | 3.84 |
| Hbegf | 3.84 |
| Fgf7 | 3.39 |
| Tgfbr1 | 3.25 |
| Hgf | 3.17 |
| Hgfac | 2.92 |
| Ngfb | 2.86 |
| Vegfa | 2.74 |
| Fgfr | 2.53 |
| Igfbp6 | 2.34 |
| Pdgf | 2.34 |
| Igfbp3 | 2.24 |
| Bmp1 | 2.14 |
| Epithelial phenotype/ | |
| solute carriers | Fold increase |
| E-cadherin | 2.42 |
| Laminin-1 | 2.09 |
| Slc7a8 | 11.87 |
| Slc24a3 | 8.76 |
| Slc11a1 | 6.68 |
| Slc7a11 | 6.15 |
| Slc16a9 | 4.06 |
| Gene names | Fold decrease |
| Erythroid associated | 312.5 |
| factor | |
| Ig heavy chain | 285.7 |
| Pre-B lymphocyte | 101.2 |
| gene 3 | |
| ATP binding casette b4 | 6.15 |
| CD2 antigen | 5.35 |
| Myeloperoxidase | 5.26 |
| DNA | 4.35 |
| methyltransferase 3B | |
| Kruppel-like factor 4 | 3.81 |
| (Klf4) | |
| Histone deacetylase 7 | 3.38 |
| E2F transcription | 3.16 |
| factor 2 | |

All of the methods disclosed and claimed herein can be made and executed without undue experimentation in light of the present disclosure. While the compositions, systems and methods of this invention have been described in terms of particular embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,843,780
Adams et al., Nature, 439:599-603, 2006.
Akashi et al., Blood, 101:383-389, 2003.
Akashi et al., Blood, 5:5, 2002.
Alexanian, J. Cellular Biochem., 100:362-371, 2007.
Backs and Olson, Circul. Res., 98: 15-24, 2006.
Bain et al., Dev. Biol., 168:342-357, 1995.
Balasubramaniyan et al., Neuroscience, 143:939-951, 2006.
Becker et al., Experim. Heatology, 27:533-541, 1999.
Bi et al., J. Am. Soc. Nephroi., 18:2486-2496, 2007.
Bonig et al., Blood, 107:79-86, 2006.
Bonventre, Kidney Intl., 43:1160-1178, 1993.
Bradley et al., Nature, 309:255-256, 1984.
Cain et al., Differentiation, 76(8):831-42, 2008.
Calvi et al., Nature, 425: 841-846, 2003.
Carey and La Thangue, Current Opin. Pharmacol., 6:369-375, 2006.
Cerny and Quesenberry, J. Cellular Physiology, 201:1-16, 2004.
Chang S et al., Cell, 126: 321-334, 2006.
Chen et al., J. Am. Soc. Nephroi., 18:456 A, 2007.
Chute, Curr. Opin. Hematol., 13:399-406, 2006.
Doetschman et al., J. Embryol. Exp. Morph., 87:27-45, 1985.
Dooner et al., Blood Cells Molec. Dis., 32:47-51, 2004.
Dreux et al., Atherosclerosis, 186 (1):38-53, 2006.
Duffield and Bonventre, Kidney Intl., 68:1956-1961, 2005.
Duffield et al., J. Clin. Invest., 115:1743-1755, 2005.
Evans et al., Nature, 292:154-156, 1981.
Fang et al., J. Am. Soc. Nephroi., 16:1723-1732, 2005.
Gallagher and Lyon, In: Proteoglycans: structure, biology and molecular interactions, Marcel Dekker Inc., NY, 27-59, 2000.
Gearing et al., Biotechnology, 7:1157-1161, 1989.
Gonzalez et al., Kidney Int., 61 (4): 1322-31, 2002.
Gould et al., Kidney Int., 61(1):51-60, 2002.
Gray et al., Intl. J. Oncology, 24:773-795, 2004.
Gupta et al., Kidney Intl., 62:1285-1290, 2002.
Gutierrez-Ramos et al., Proc. Nat. Acad. Sci. USA, 89:9111-9175, 1992.
Harris et al., Exp. Cell. Res., 284(1):2-13, 2003.
Hollnagel et al., J. Biol. Chem., 274:19838-19345, 1999.
House et al., J. Bone Miner Res., 12:1959-1970, 1997.
Humphreys et al., Cell Stem Cells, 2:284-291, 2008.
Ianus et al., J. Clin. Invest., 111:843-850, 2003.
Ikuta et al., Proc. Natl. Acad. Sci. USA, 89:1502-1506, 1992.
Ito et al., J. Am. Soc. Nephrol., 12:2625-2635, 2001.
Jaenisch and Bird, Nature Genetics, 33(Suppl):245-254, 2003.
Jaenisch and Young, Cell, 132:567-582, 2008.
James et al., Develop. (Cambridge, England), 133:2995-3004, 2006.
Jang et al., Nat. Cell Biol., 6:532-539, 2004.
Jenuwein and Allis, Science, 293:1074-1080, 2001.
Kale et al., J. Clin. Invest., 112:42-49, 2003.
Kaplan et al., Trends Molec. Med., 13:72-81, 2007.
Kiel et al., Cell, 121:1109-1121, 2005.
Kim and Dressler, J. Am. Soc. Nephrol., 16(12):3527-34, 2005.
Kitisin et al., Sci STKE., 2007(399), 2007.
Komaki et al., J. Cell. Sci., 117(8):1457-1468, 2004
Kondo and Raff, Genes & Develop., 18:2963-2972, 2004.
Kopp et al., Physiology (Bethesda), 20:349-356, 2005.
Kos et al., J. Clin. Invest., 111:1021-1028, 2003.
Kretchmar and Conover, Transplantation, 8:576-581, 1969.
Kucia et al., Stem cells, 23:879-894, 2005.
Laird et al., Cell, 132:612-630, 2008.
Lambert et al., J. Experim. Med., 197:1563-1572, 2003.
Lange et al., Kidney Intl., 68:1613-1617, 2005.
Lapidot et al., Blood, 106:1901-1910, 2005.
Lee et al., Cell, 125:301-313, 2006a.
Lee et al., Molec. Cancer Therap., 5:3085-3095, 2006b.
Li et al., J. Am. Soc. Nephrol., 18:3067-3077, 2007.
Liano and Pascual, Semin. Nephrol., 18:541-550, 1998.
Lin et al., J. Am. Soc. Nephroi., 14:1188-1199, 2003.
Lin et al., J. Clin. Invest., 115:1756-1764, 2005.
Little et al., Gene Expr. Patterns, 7:680-699, 2007.
Luskey et al., Blood, 80:396-402, 1992.
Maherali et al., Stem Cells, 1:55-70, 2007.
McGrane, J. Nutritional Biochem., 18:497-508, 2007.
McKinsey et al., Proc. Natl. Acad. Sci. USA, 97:14400-14405, 2000.
Medvinsky and Dzierzak, Cell, 86:897-906, 1996.
Mendelsohn et al., Develop. (Cambridge, England), 126:1139-1148, 1999.
Meshorer et al., Develop. Cell, 10:105-116, 2006.
Mitsiades et al., Proc. Natl. Acad. Sci. USA, 101:540-545, 2004.
Morigi et al., J. Am. Soc. Nephrol., 15:1794-1804, 2004.
Nagy et al., Development, 110:815-821, 1990.
Nemeth et al., J. Pharm. Experim. Therap., 299:323-331, 2001.
Nilsson et al., Blood, 97:2293-2299, 2001.
Okita et al., Nature, 448(7151):313-7, 2007.
Olszak et al., J. Clin. Invest., 105:1299-1305, 2000.
Orlic et al., Nature, 410:701-705, 2001.
Osafune et al., Dev. Grow. Differen., 44:161-167, 2002.
Papayannopoulou and Craddock, Acta Haematol., 97:97-104, 1997.
Park et al., Blood, 99:488-498, 2002.
Passegue et al., J. Experim. Med., 202:1599-1611, 2005.
PCT Appln. WO 99/61589
Peart et al., Proc. Natl. Acad. Sci. USA 102:3697-3702, 2005.
Pease et al., Dev. Biol., 141:344-352, 1990a.
Pease et al., Exp. Cell Res., 190:209-211, 1990b.
Petersen, J. Appl. Physiol., 98(4):1154-62, 2005.
Poulsom et al., J. Pathol., 195:229-235, 2001.
Quesenberry, Curr. Opin. Hematol., 13:216-221, 2006.
Reddy et al., Blood, 90:2293-2299, 1997.
Reubinoff et al., Nature Biotech., 18:399, 2000.
Riccardi et al., Amer. J. Physiology, 271: F951-956, 1996.
Robbins et al., J. Biol. Chem., 265:11905-11909, 1990.
Robertson, In: Teratocarcinomas and Embryonic Stem Cells: A Practical Approach, Washington, D.C.: IRL Press, 1987.
Schmitt et al., Genes and Development, 5:728-740, 1991.
Schmittwolf et al., EMBO J., 24:554-566, 2005.
Schofield, Blood Cells, 4:7-25, 1978.
Schrier et al., J. Clin. Invest., 114:5-14, 2004.
Shang et al., Cell, 103:843-852, 2000.
Slager et al., Dev. Genet., 14:212-224, 1993.
Smith et al., Nature, 36:688-690, 1988.
Snykers et al., BMC Develop. Biology, 7:24, 2007.
Spangrude et al., Science, 241:58-62, 1988.
Sugimoto et al., Proc. Natl. Acad. Sci. USA 103:7321-7326, 2006.
Supavekin et al., Kidney Intl., 63:1714-1724, 2003.
Takahashi and Yamanaka, Cell, 126:663-676, 2006.
Takahashi et al., Cell, 131:861-872, 2007.
Tayaramma et al., Stem Cells, 24:2858-2867, 2006.
The Kidney, Brenner and Floyd (Eds.), WB Saunders Co., Philadelphia, 2001.
Thomson et al., Proc. Natl. Acad. Sci. USA, 92:7844, 1995.
Thomson et al., Science, 282:1145, 1998
Thomson, Curr. Top. Dev. Biol., 38:133, 1998.
Till et al., Radix. Res., 14:213-222, 1961.
Togel et al., Am. J. Physiol. Renal. Physiol., 289:F31-42, 2005a.
Togel et al., Am. J. Physiol. Renal. Physiol., 292(5):F1626-35, 2007.
Togel et al., Kidney Intl., 67:1772-1784, 2005b.
Van Lint et al., Gene Express., 5:245-253, 1996.
Verdin et al., Trends Genet., 19:286-293, 2003.
Vigushin et al., Clin. Cancer Res., 7:971-976, 2001.
Vos et al., Cell Tissue Kinetics, 5:467-479, 1972.
Vukicevic et al., J. Clin. Invest., 102:202-214, 1998.
Watanabe et al., Genes Develop., 18:889-900, 2004.
Wernig et al., Nature, 448(7151):318-24, 2007.
Wiles et al., Development, 111:254-267, 1991.
Wiles et al., Exp. Cell. Res., 247:241-248, 1999.
Williams et al., Nature, 336:684-687, 1988.
Wilpshaar et al., Blood, 96: 2100-2107, 2000.
Wineman et al., Blood, 81:365-372, 1993.
Wobus et al., Biomed. Biochim. Acta, 47:965-973, 1988.
Wolf et al., Exp. Hematol., 21:614-622, 1993.
Xi and Xie, Science, 310:1487-1489, 2005.
Xue et al., J. Am. Soc. Nephrol., 17:1135-1142, 2006.
Yagil et al., Amer. J. Physiology, 256:F456-461, 1989.
Yeom et al., Develop. (Cambridge, England), 122:881-894, 1996.
Yu et al., Science, 318(5858):1917-20, 2007.
Zhang et al., Cell, 110:479-488, 2002.
Zhang et al., Nature, 425:836-841, 2003.

What is claimed is:

1. A method of generating an induced renal cell comprising the steps of:
a) obtaining lineage an undifferentiated hematopoietic stem and/or progenitor cell; and
b) incubating the hematopoietic stem and/or progenitor cell for a period sufficient to produce an induced renal cell in the presence of:
at least a histone deacetylase (HDAC) inhibitor,
(ii) at least one nephrogenic factor selected from the group consisting of a first agent capable of activating a retinoic acid pathway, a second agent capable of activating an activin-A pathway, and a third agent capable of activating a BMP7 pathway, and,
(iii) an epithelial growth factor.

2. The method of claim 1, wherein the nephrogenic factor comprises at least two of the group consisting of said first agent, said second agent and said third agent.

3. The method of claim 1, wherein the nephrogenic factor comprises said first agent, said second agent and said third agent.

4. The method of claim 1, wherein said first agent is retinoic acid.

5. The method of claim 1, wherein said second agent is activin-A.

6. The method of claim 1, wherein said third agent is BMP7.

7. The method of claim 1, wherein the nephrogenic factor comprises retinoic acid, activin-A and BMP7.

8. The method of claim 1, wherein the HDAC inhibitor is selected from the group consisting of a small molecule HDAC inhibitor, a HDAC siRNA, and a HDAC antibody.

9. The method of claim 8, wherein the HDAC inhibitor is selected from the group consisting of hydroxamic acid, cyclic peptide, benzamide, electrophilic ketone, and aliphatic acid.

10. The method of claim 9, wherein the hydroxamic acid is selected from the group consisting of trichostatin A (TSA), vorinostat or suberoylanilide hydroxamic acid (SAHA), CBHA, belinostat/PXD-101, ITF2357 and LBH-589.

11. The method of claim 9, wherein the cyclic peptide is selected from the group consisting of PCI-24781 and depsipeptide (FK-228).

12. The method of claim 9, wherein the benzamide is selected from the group consisting of MS-275, CI-994 and MGCD0103.

13. The method of claim 9, wherein the aliphatic acid is selected from the group consisting of valproic acid, butyrate, phenylbutyrate and AN-9.

14. The method of claim 10, wherein the HDAC inhibitor is TSA.

15. The method of claim 8, wherein the siRNA is a HDAC7 siRNA and/or a HDAC9 siRNA.

16. The method of claim 1, wherein the epithelial growth factor is selected from the group consisting of EGF, IGF-1 and HGF.

17. The method of claim 16, wherein the epithelial growth factor comprises two or more of Epidermal growth factor (EGF), Insulin-like growth factor 1 (IGF-1) and Hepatocyte growth factor (HGF).

18. The method of claim 1, wherein the method further comprises incubating the hematopoietic stem or progenitor cell with at least a cytokine.

19. The method of claim 18, wherein the cytokine is selected from the group consisting of IL-3, IL-6 and stem cell factor.

20. The method of claim 18, wherein the cytokine comprises two or more of IL-3, IL-6 and stem cell factor.

21. The method of claim 1, wherein the method further comprises step (c) of isolating an induced renal cell.

22. The method of claim 21, wherein the isolated induced renal cell is incubated with a calcium-sensing receptor (CaR) inhibitor.

23. The method of claim 22, wherein the CaR inhibitor is a CaR antibody, a CaR siRNA or a small molecule CaR inhibitor.

24. The method of claim 1, wherein the lineage undifferentiated hematopoietic stem and/or progenitor cell is an umbilical cord stem cell.

25. An induced renal cell generated according to claim 1.

26. A method of treating a renal disease or injury comprising administering an effective amount of the induced renal cell of claim 1 to a subject having a renal disease or injury.

27. The method of claim 26, wherein the renal disease or injury is acute kidney injury, chemical or toxin injury, urinary tract obstruction, acute tubular necrosis and apoptosis, glomerular injury and/or inflammation, early dysfunction of kidney transplant, or chronic renal failure.

28. The method of claim 26, wherein the renal disease or injury is acute ischemic/hypoxic kidney injury.

29. A culturing system comprising a incubator containing a culture medium, where the culture medium comprises:
a) a nephrogenic factor selected from the group consisting of a first agent capable of activating a retinoic acid pathway, a second agent capable of activating an activin-A pathway, and a third agent capable of activating a BMP7 pathway; and
b) a HDAC inhibitor.

30. The culturing system of claim 29, wherein the nephrogenic factor is selected from the group consisting of retinoic acid, activin-A and BMP7.

31. The culturing system of claim 29, wherein the nephrogenic factor comprises two or more of retinoic acid, activin-A and BMP7.

32. The culturing system of claim 29, wherein the nephrogenic factor comprises BMP7.

33. The culturing system of claim 29, wherein the nephrogenic factor comprises retinoic acid.

34. The culturing system of claim 29, wherein the HDAC inhibitor is selected from the group consisting of a small molecule HDAC inhibitor, a HDAC siRNA, and a HDAC antibody.

35. The culturing system of claim 29, wherein the HDAC inhibitor is selected from the group consisting of hydroxamic acid, cyclic peptide, benzamide, electrophilic ketone, and aliphatic acid.

36. The culturing system of claim 35, wherein the HDAC inhibitor is selected from the group consisting of the HDAC inhibitor is selected from the group consisting of trichostatin A (TSA), vorinostat or suberoylanilide hydroxamic acid (SAHA), CBHA, belinostat/PXD-101, ITF2357, LBH-589, PCI-24781, depsipeptide (FK-228), MS-275, CI-994, MGCD0103, valproic acid, butyrate, phenylbutyrate and AN-9.

37. The culturing system of claim 35, wherein the HDAC inhibitor is TSA.

38. The culturing system of claim 29, further comprising a hematopoietic stem and/or progenitor cell.

39. The culturing system of claim 29, further comprising an induced renal cell.

40. A method of treating one or more symptoms of renal disease or injury comprising administering to a human subject in need thereof insulin-like growth factor 1 (IGF-1), human growth factor (HGF), vascular endothelial growth factor α (VEGFα) and early growth response 1 (EGR-1).

41. The method of claim 40, wherein administering comprises intravenous, intra-arterial or intraperitoneal administration.

42. The method of claim 40, wherein the renal disease or injury is selected from acute kidney injury, chemical or toxin injury, urinary tract obstruction, acute tubular necrosis and apoptosis, glomerular injury and/or inflammation, early dysfunction of kidney transplant, acute ischemic/hypoxic kidney injury or chronic renal failure.

43. The method of claim 40, further comprising subjecting said subject to dialysis.

44. The method of claim 40, wherein administration is via bolus injection.

45. The method of claim 40, wherein administration is via continuous administration.
